(19) United States
(12) Patent Application Publication
(10) Pub. No.: US 2016/0347861 A1
(43) Pub. Date: Dec. 1, 2016

(54) REDUCED SATURATED FATTY ACID PROFILES OF RUMINANTS

(71) Applicants: The United States of America, as represented by the Secretary of Agriculture, Washington, DC (US); The Texas A & M University System, College Station, TX (US)

(72) Inventors: Robin C Anderson, College Station, TX (US); David J. Nisbet, Bryan, TX (US); Holly D. Edwards, Richmond, TX (US)

(21) Appl. No.: 15/161,367

(22) Filed: May 23, 2016

Related U.S. Application Data
(60) Provisional application No. 62/167,193, filed on May 27, 2015.

(51) Int. Cl. C07K 16/40; A61K 39/02; A61K 39/05

(52) U.S. Cl. CPC C07K 16/40; A61K 39/05; A61K 39/0208; A61K 2039/552

(57) ABSTRACT

Methods for reducing the amount of ruminal saturation of unsaturated fatty acids and poly-unsaturated fatty acids in a ruminant by administering compositions that interfere and reduce lipolysis and/or biohydrogenation are provided. In one embodiment, the composition is antibodies that bind to immunogens involved in lipolysis and/or biohydrogenation. In another embodiment, the composition is an immunogenic composition containing immunogens involved in lipolysis and/or biohydrogenation. Method for producing the antibodies are also provided.

### CROSS-REFERENCE TO RELATED APPLICATIONS

This application claims the benefit under 35 U.S.C. §119(e) to U.S. Patent Application 62/167,193 filed on May 27, 2015, the contents of which are incorporated herein by reference in its entirety.

### BACKGROUND OF THE INVENTION

Summary of the Invention

This invention relates to a method of improving the fatty acid profile of ruminants, especially in the milk and muscle tissue of the animals. This invention also relates to reducing the amount of saturated fats in the meat and milk of ruminants by reducing or inhibiting the lipolysis and/or biohydrogenation of unsaturated or poly-unsaturated fats in the rumen of the animals. This invention further relates to the use of antibodies either produced by a ruminant or ingested by a ruminant to inhibit or reduce the activity of ruminal bacteria and/or enzymes and/or cofactors involved in lipolysis and/or biohydrogenation of unsaturated or poly-unsaturated fats in the rumen of the animals.

Summary of Prior Art

Dietary fats and oils represent a significant percentage of the daily caloric intake for humans in the United States, comprising >33% of total calories Ursin, V. M., J. Nutr. 133: 4271-4274 (2003)). The replacement of trans fatty acids with unsaturated fatty acids has been shown to be one of the most effective measures for improving blood lipid profiles (Mensink, et al., Am. J. Clin. Nutr. 77:1146-1155 (2003)). The quality of dietary lipids for human consumption has also been shown to be of importance for the development of insulin resistance and related metabolic syndromes, such as but not limited to, blood lipid disorders, hypertension, propensity for thrombus formation, abdominal obesity and type II diabetes mellitus (DeFronzo and Ferrannini, Diabetes Care 14:173-194 (1991)). Trans fatty acids are naturally produced in the rumen of animals because of incomplete biohydrogenation. Recent changes in legislation have prohibited the inclusion of synthetic trans fatty acids in any food or drink for human consumption.

Current recommendations limit total fat intake to <35% of daily calories, 7% of that comprised of saturated fatty acids and the remainder from monounsaturated and polyunsaturated fatty acids (Lichtenstein, et al., Circulation 114:82-96 (2006)). Saturated and trans fatty acids make up the majority of lipids found in the meat and milk derived from ruminal animals. Milk and milk products are the major sources of fat in most diets, accounting, on average, for about 30% of the total fat and 40% of the saturated fat intake in the U.K. (Mansbridge and Blake, Brit. J. Nutr. 78:S37-S47 (1997)), Belgium (Demeyer, et al., Meat quality in double muscled animals. In composition of meat in relation to processing. In: K. Lindström, I. Hansson and E. Wiklund (eds.) Nutritional and Sensory Quality p 95-102. (1995)) and Germany (Flachowsky and Jahreis, Fett/Lipid. 99:106-115 (1997)). Beef is estimated to contribute only about 5% of the total fat intake, but is regarded as a rich source of saturated fat (Demeyer and Doreau, Proceedings of the Nutrition Society 58:593-607 (1999)).

Lactating dairy cows, on average, consume around 300 g of linoleic acid (18:2n6) daily, of this only about 40 g remain unsaturated and reaches the small intestine intact (Jenkins and Bridges, Eur. J. Lipid Sci. Technol. 109:778-789 (2007)). Absorption of dietary lipids occurs primarily in the small intestine of ruminants. Lipolysis and biohydrogenation processes occur in the rumen as a result of microbial metabolic activity (Shorland, et al., Nature 175:1129-1130 (1955); Viviani, R., Adv. Lipid Res. 8:267-346 (1970)). Microorganisms are responsible for reducing the double bonds found in unsaturated fatty acids leaving the carbons free to attach to hydrogen molecules; this effectively transforms unsaturated fats into saturated fats (Doreau and Chilliard, Brit. J. Nutr. 78:515-535 (1997)). Biohydrogenation is a detoxification process, necessary for bacteria to escape from the bacteriostatic effects of unsaturated fatty acids (Maia, et al., BMC Microbial. 10:52 (2010)). The prevention of biohydrogenation is important because incomplete biohydrogenation results in trans fatty acid production whereas complete biohydrogenation results in the production of saturated fatty acids. In order for biohydrogenation to occur, free fatty acids must first be hydrolyzed from their triacylglycerol precursors, a process known as lipolysis (Harfoot and Hazlewood, Lipid metabolism in the rumen. In: P. N. Hobson and C. S. Stewart (eds.) The Rumen Microbial Ecosystem p 382-426. Chapman & Hall, London, UK (1997)). Extracellular lipases, expressed by bacteria, catalyze the hydrolysis of mono-, di- and triacylglycerols to free fatty acids and glycerol (Aravindan, et al., Indian J. Biotechnol. 6:141-148 (2007); Pandey, et al., Biotechnol. Appl. Biochem. 29:119-131 (1999)).

U.S. Pat. No. 7,344,713 disclosed the generation of IgY antibodies that bind to swine lipase. The anti-swine lipase antibodies either only slightly reduced, or, surprisingly, enhanced purified swine lipase activity when they were combined in-vitro. Administration of the anti-swine lipase antibodies to mice resulted in slightly less weight in than in negative control mice. No experiments were conducted to determine the effect of administration of the anti-lipase antibodies to ruminants.

As early as 2008, it was demonstrated that chicken antibodies (IgY) generated against each of Anaerovibrio lipolytica 5S, Butyrivibrio fibrisolvens strain H17C, Clostridium chauvoei, and Propionibacterium acnes were able to reduce the amount of free fatty acids in bovine ruminal fluids in-vitro. See, e.g., Krueger, et al., J. Anim. Sci. 86 E-Suppl. 2:87-88 (2008) and Krueger, et al., Microb. Ecol. 57:562-588 (2009).

As recently as April, 2013, IgY antibodies were generated against, individually, A. lipolyticus 5S, B. fibrisolvens strain H17C, P. avidum, P. acnes, and purified lipase, and then assessed in an in-vitro assay for reduction of bacterial lipolysis of olive oil. Some antibodies were better than others in reducing lipolysis in the in-vitro assay. The effectiveness of the anti-B. fibrisolvens antibodies in reducing biohydrogenation also was examined, in-vitro, by culturing B. fibrisolvens strain H17C in either linoleic acid (18:2n-6) or α-linolenic acid (18:3n-3) in an anerobic assay buffer. The anti-B. fibrisolvens antibodies did not reduce biohydrogenation of linoleic acid yet reduced biohydrogenation of α-linolenic acid (Edwards, et al., Plains Nutrition Council Spring Conference, April 2013).

Despite these prior art experiments, nobody has examined the in-vivo effect on ruminal lipid profiles of oral administration of antibodies against lipase and/or ruminal bacteria to ruminants. Nor, has anybody determined if ruminants could generate antibodies to lipase and/or ruminal bacteria and the in-vivo effect of those antibodies on ruminal lipid profiles. Further, a need exists for reducing naturally occurring trans fatty acids in ruminal meats and milk. This invention fulfills that need, resulting in a healthier fatty acid profile in ruminal meat and milk.

### BRIEF DESCRIPTION OF THE INVENTION

It is an object of this invention to use antibodies to reduce or inhibit lipolysis and/or biohydrogenation (which includes isomerization and saturation) in a ruminant's rumen so that fewer saturated fatty acids and/or trans fatty acids are produced. As such, it is another object of this invention to increase the amount of unsaturated fatty acids, poly-unsaturated fatty acids, galactolipids, triacylglycerides, and phospholipids that pass through the rumen into the small intestine and are absorbed into the blood stream where they are incorporated into the ruminant's milk and/or muscle. It is a further object of this invention to administered antibodies made by another animal to the ruminant. These antibodies bind to one or more ruminal bacteria, one or more enzymes, and/or one or more co-factors involved in lipolysis and/or biohydrogenation, thereby reducing the activity of the bacteria, the enzymes, and/or co-factors, and thus reducing the amount of biohydrogenation and/or lipolysis that occurs in the treated ruminant, compared to an untreated ruminant. In one embodiment, the antibodies reduce the growth and/or replication of ruminal bacteria involved in lipolysis and/or biohydrogenation.

It is an object of this invention to have an immunogenic composition containing a pharmaceutically acceptable carrier, optionally an adjuvant, and (i) one or more of these ruminal bacteria, (ii) one or more enzymes and/or fragments thereof, (iii) one or more co-factors and/or fragments thereof, or (iv) a combination thereof, all of which are involved in lipolysis and/or biohydrogenation. It is another object of this invention to administer this immunogenic composition to an animal in order to induce the animal to produce antibodies to the immunogens of the immunogenic composition. It is a further object of this invention to collect these antibodies (either purified or unpurified) and administer the antibodies to the ruminant to produce the desired effects described above.

It is another object of this invention to administer the immunogenic composition described in the preceding paragraph to a ruminant to induce the ruminant to produce antibodies that bind to the immunogen(s) in the immunogenic composition. It is a further object of this invention that the antibodies so produced bind to the immunogen(s) and reduce biohydrogenation and/or lipolysis in the rumen, thereby producing the desired effects described above.

### BRIEF DESCRIPTION OF THE FIGURES

FIG. 1 shows the major steps in ruminal biohydrogenation of dietary unsaturated lipids. Hatched arrows represent areas that are reduced or inhibited by the invention described herein. In FIG. 1, “CLA” refers to “conjugated linoleic acid”.

FIG. 2A is the DNA sequence (SEQ ID NO: 1) for pro-lipase (gheA). FIG. 2B is the amino acid sequence (SEQ ID NO: 2) for pro-lipase (gheA).

FIG. 3A is the DNA sequence (SEQ NO: 3) for mature lipase. FIG. 3B is the amino acid sequence (SEQ ID NO: 4) for mature lipase.

### DETAILED DESCRIPTION OF THE INVENTION

Lipase-expressing bacteria and hydrogenating bacteria found in the rumen play a major role in the composition and alteration of ruminal lipids and ultimately what is deposited in the animal's muscles and milk. This invention changes those interactions between the ruminal bacteria, enzymes, and/or co-factors, thereby positively impacting the lipid profiles in the rumen, and thus in the small intestine, blood stream, and the rest of the ruminant. One method for protecting unsaturated fatty acids is the prevention or reduction or inhibition of free carboxyl group production on ruminal lipids, a requirement for biohydrogenation, by reducing the activity of ruminal bacterial lipases, co-factors, and/or other enzymes involved in lipolysis, and/or reducing the growth of ruminal bacteria involved in lipolysis. Another method is protecting the free fatty acids from the initial stages of biohydrogenation via reducing the activity of ruminal bacterial saturase, lipase, co-factors, and/or other enzymes involved in biohydrogenation, and/or reducing the growth of ruminal bacteria involved in biohydrogenation. When lipolysis and biohydrogenation of unsaturated fatty acids and poly-unsaturated fatty acids are reduced or inhibited, then the ruminant has higher levels of triacylglycerides, phospholipids, and galactolipids that can pass through the rumen into the intestine and then be absorbed into the blood stream. Another benefit is that ruminants can be fed higher amounts of oils (such as, but not limited to, soybean oil, olive oil, linseed/flaxseed oil) without adverse effects.

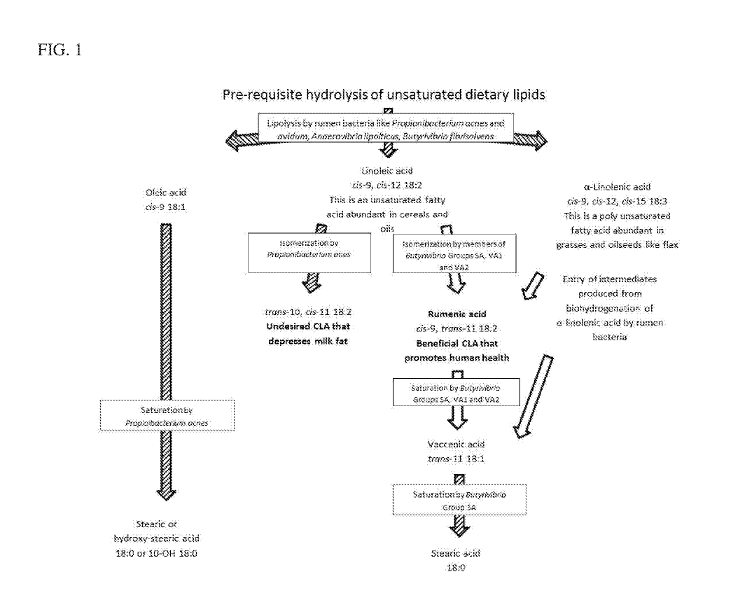

Ruminal biohydrogenation (i.e., saturation of unsaturated fatty acids and poly-unsaturated fatty acids by rumen bacteria) include three major steps. As shown in FIG. 1, these steps involve “pre-requisite hydrolysis” (also referred to as “lipolysis”) of lipids to generate free unsaturated fatty acids, the isomerization of the free unsaturated fatty acids into structural forms accessible for saturation, and a sequential series of saturations which yield fully saturated fatty acids and trans fatty acids. In lipolysis, extracellular lipases catalyze the hydrolysis of triacylglycerides, phospholipids, and galactolipids contained in ruminant feed, releasing oleic acid, linoleic acid and linolenic acid. Extracellular lipases are either free floating in the rumen or bound to the exterior surface of lipase-producing bacteria (e.g., Propionibacterium acnes, P. avidum, Anaerovibrio lipolyticus, and numerous Butyrivibrio species). Isomerization of linoleic acid (cis-9, cis-12 18:2) and α-linolenic acid (cis-9, cis-12, cis-15 18:3), the next step, is catalyzed by a variety of bacteria. Butyrivibrio groups SA, VA1, and VA2 isomerize the majority of linoleic acid (cis-9, cis-12 18:2) into beneficial conjugated linolenic acid, cis-9, trans-11 18:2 (also called rumenic acid). See, Paillard, et al. Antonie van Leeuwenhook 91:417-422 (2007). In FIG. 1, “CLA” refers to “conjugated linolenic acid”. However, under certain conditions, P. acnes isomerizes linoleic acid into an undesired CLA isomer (trans-10, cis-11 18:2) which depresses de novo milk fat synthesis in cows. McKain, et al., Microbiology 156:579-588 (2010). Approximately 50% of the fat in milk is synthesized by the mammary glands; the other 50% of fat in milk are dietary fatty acids. The third step is sequential saturation of the isomerized lipids. Saturation of the beneficial CLA, cis-9, trans-11 18:2, which is a main intermediate within the rumen, to trans-11 18:1 (commonly named vaccenic acid), is accomplished by Butyrivibrio groups SA, VA1 and VA2 (groups VAT and VA2 are so named for their contribution to vaccenic acid production). Vaccenic acid is also an intermediate in the rumen, being reduced in a final saturation step to stearic acid (18:0) exclusively by Butyrivibrio group SA (so named for their exclusive contribution to stearic acid production).

While not wishing to be bound to any particular hypothesis, it is believed that the invention described herein interferes with the steps represented by hatched arrows in FIG, 1. By reducing or preventing lipolysis and/or biohydrogenation, this invention promotes ruminal passage, intestinal absorption, and incorporation of health-promoting unsaturated fatty acids and beneficial CLA (cis-9, trans-11 18:2) into ruminant meat and milk. The invention also prevents production of undesired CLA (trans-10, cis-11 18:2) which decreases the value of milk produced because of the lower fat content. Not wishing to be bound to any particular hypothesis, the anti-lypolytic antibodies described herein bind to their lipolytic immunogen and interfere with the enzymes and/or bacteria necessary to catalyze hydrolysis. Because biohydrogenation requires the fatty acid molecule to have a free, unhindered carboxyl group, the anti-lypolytic antibodies effectively protect most of the unsaturated fatty acids in their bound (lipid) form, thereby increasing their passage to the small intestine where they are digested and absorbed similar to monogastric animals which lack biohydrogenation.

Furthermore, antibodies generated against P. acnes and/or their enzymes and/or their co-factors (either by the ruminant or by another animal and administered to the ruminant) bind to P. acnes, their enzymes, and/or their co-factors, thereby reducing or inhibiting the undesired isomerization process which generates CLA trans-10, cis-11 i8:2. Also, antibodies generated against Butyrivibrio group SA and/or their enzymes and/or co-factors can reduce or prevent the final saturation of vaccenic acid (trans-11 18:1) to stearic acid (18:0), thereby allowing vaccenic acid to pass from the rumen to the small intestine where vaccenic acid is assembled into micelles and absorbed into the blood stream. Once absorbed, vaccenic acid is nearly as beneficial and valuable as rumenic acid in dairy animals, because vaccenic acid is transported to the mammary gland where it is converted back to the beneficial CLA, cis-9, trans-11 18:2 (rumenic acid) by delta-9 desaturase activity in the mammary gland. Vaccenic acid is also converted back to rumenic acid, albeit at a lesser amount, by delta-9 desaturase activity in the liver. It is also hypothesized that antibodies generated against Butyrivibrio group SA and/or their enzymes and/or co-factors also inhibit Butyrivibrio group SA's contribution to isomerization and the early saturation steps, but have no or little effect on the activity of Butyrivibrio groups VA1 and VA2. Concerning oleic acid saturation, antibodies against P. acnes and/or its enzymes and/or its co-factors reduce oleic acid's conversion to stearic or hydroxyl stearic acid, thus promoting passage, absorption, and incorporation of oleic acid into ruminant produced meat and milk. Thus, the invention also reduces the production of saturated fatty acids compared to untreated animals.

A ruminant is a mammal that acquires nutrients from plant-based food by fermenting it in a specialized stomach (a four-compartment stomach) prior to digestion, principally through bacterial actions. Rumen, reticulum, omasum, and abomasum are the four parts of the ruminant's stomach. In the rumen and reticulum, food mixes with saliva and separates into solids and liquid material. The solids clump together to form the cud which is regurgitated and chewed to completely mix it with saliva and to break down the particle size. Fiber, especially cellulose and hemi-cellulose, is primarily broken down in the rumen and reticulum by microbes (mostly bacteria, as well as some protozoa and fungi) into acetic acid, propionic acid and butyric acid (volatile fatty acids). The bacteria in the rumen convert the unsaturated fatty acids and poly-unsaturated fatty acids into saturated fatty acids. Volatile fatty acids which are short-chained fatty acids are absorbed in the rumen. Longer chain fatty acids which include saturated and unsaturated fatty acids are absorbed in the intestine. Proteins and non-structural carbohydrates (pectin, sugars, starches) are also fermented in the rumen and reticulum. The rumen and reticulum are sometimes described as one organ because digesta moves back and forth between them. Some people use “reticulorumen” to refer to these two chambers. Not wishing to be bound to any particular hypothesis, it is believes that the majority of lipolysis and biohydrogenation occurs in the rumen, with minor amounts in the reticulum. For the purposes of this invention, “rumen” is used to cover the areas of reticulorumen in which biohydrogenation and lipolysis occurs. Non-limiting examples of ruminants are cattle, buffalo, goats, sheep, giraffes, yaks, deer, and antelope.

For the purposes of this invention, Galliformes is the order of birds that include grouse, ptarmigan, capercaillie, partridges, pheasants, quails, turkeys, chickens, and peacocks. These animals are primarily grain-eating, heavy-bodied, ground-nesting birds, capable of only short, rapid flights. In one embodiment, a female chicken (Gallus gallus domestica), either in the adult stage or at the age of at least 17-19 weeks, is used for generating IgY antibodies. In another embodiment, any of the female birds in Galliformes can be used for generating IgY antibodies. For the purposes of this invention, a hen includes any female bird in Galliformes.

One invention described herein involves reducing lipolysis in the rumen and thus the amount of fatty acids in milk and meats by administering to the ruminant a lipolysis-inhibiting agent in an amount effective to reduce lipolysis in the treated ruminant compared to the amount of lipolysis in an untreated ruminant. This lipolysis-inhibiting agent is, in one embodiment, an anti-lipolytic antibody which binds to an epitope on (i) lipase, (ii) bacterial lipase, (iii) another enzyme(s) involved in ruminal lipolysis, (iv) a co-factor involved in ruminal lipolysis, and/or (v) at least one ruminal bacterial species that produces a lipolytic enzyme and/or lipolytic co-factor. One can administer the anti-lipolytic antibody to the ruminant orally, i.e., in animal feed or water or other composition that the ruminant ingests. In this manner, the anti-lipolytic antibody is swallowed and transported to the rumen. Of course, other modes of administration of this anti-lipolytic antibody are possible so long as the anti-lipolytic antibody reaches the rumen, interacts with its immunogen (an anti-lipolytic immunogen), and reduces the amount of lipolysis in the rumen. The anti-lipolytic antibody can be coated (contained within a protective shell) or uncoated. In one embodiment, the anti-lipolytic antibody is IgY obtained from hens. In another embodiment, the anti-lipolytic antibody are any other antibody type (i.e., IgA, IgM, IgG, IgD, and IgE) obtained from an animal. The anti-lipolytic antibody, after administration to the ruminant, binds to the lipolytic immunogen and reduces the rate of lipolysis in the rumen (compared to lipolysis rates in the rumen of ruminants that do not receive the anti-lipolytic antibody).

To generate anti-lipolytic antibodies, an immunogenic composition is administered to an animal in an amount effective to generate an immune response. For IgY antibodies, the immunogenic composition is administered to a hen. This immunogenic composition contains a lipolytic immunogen, a pharmaceutically acceptable carrier, and optionally an adjuvant. The lipolytic immunogen can be lipase, another enzyme involved in lipolysis (lipolytic enzyme a co-factor involved in lipolysis (lipolytic co-factor), at least one species of bacteria that produces one of these compounds (lipase, other lipolytic enzymes, or co-factors), or a combination thereof. The enzymes and co-factors can be purified or unpurified. The lipolytic immunogen can also be a fragment of lipase, another lipolytic enzyme, or lipolytic co-factor (i.e., a polypeptide) so long as when the anti-lipolytic antibody binds to the lipolytic immunogen, the lipolytic immunogen's activity is reduced. Lipase-producing bacteria include, but are not limited to, Propionibacterium acnes, Propionibacterium avidum, Anaerovibrio lipolyticus, and Butyrivibrio fibrisolvens. Lipase-producing bacteria also include other ruminal bacteria that produce lipase or are involved in the production of other lipolytic enzymes or lipolytic co-factors. In one embodiment, the immunogenic composition contains a combination of any of the above described lipolytic immunogens.

In another embodiment, the lipolysis-inhibiting agent is administered directly to a ruminant. This lipolysis-inhibiting agent is an immunogenic composition containing one or more of the lipolytic immunogens (discussed above), a pharmaceutically acceptable carrier, and optionally an adjuvant. This immunogenic composition is administered to the ruminant in an amount effective to produce an immune response in the ruminant. In one embodiment, the ruminant's immune response is the production of secretory IgA antibodies that bind to the lipolytic immunogen. In another embodiment, the ruminant's immune response is the production of other types (classes) of antibodies that bind to the lipolytic immunogen. In these embodiments, the antibodies, when bound to the lipolytic immunogen, reduce the rate of lipolysis in the rumen (compared to lipolysis rate in the rumen of ruminants that do not produce the anti-lipolytic immunogen antibody).

Another invention described herein enhances ruminal passage of unsaturated fatty acids and poly-unsaturated fatty acids (collectively, “unsaturated fatty acids”, unless otherwise noted) in a ruminant. By increasing the passage of these unsaturated fatty acids through the rumen, these unsaturated fatty acids reach the small intestine, are absorbed into the blood stream, and are incorporated into the ruminant's muscle tissue and milk. Thus, a ruminant's meat and milk will have higher amounts of unsaturated fatty acids and poly-unsaturated fatty acids compared to untreated ruminants. In this invention, an unsaturated fatty acid protectant is administered to the ruminant in an amount effective to enhance the ruminal passage of unsaturated fatty acids and poly-unsaturated fatty acids. In one embodiment, this unsaturated fatty acid protectant is an anti-lipolytic antibody which binds to an epitope on (i) lipase, (ii) a bacterial lipase, (iii) another enzyme(s) involved in ruminal lipolysis, (iv) a co-factor involved in ruminal lipolysis, and/or (v) at least one ruminal bacterial species that produces a lipolytic enzyme and/or a lipolytic co-factor. One can administer the anti-lipolytic antibody to the ruminant orally, i.e., in animal teed or water or other composition that the ruminant ingests. In this manner, the anti-lipolytic antibody is swallowed and transported to the rumen. Of course, other modes of administration of this anti-lipolytic antibody are possible so long as the anti-lipolytic antibody reaches the rumen, interacts with its immunogen (an anti-lipolytic immunogen), and reduces the amount of lipolysis in the rumen. The anti-lipolytic antibody can be coated (contained within a protective shell) or uncoated. In one embodiment, the anti-lipolytic antibody can be IgY antibodies obtained from hens. In another embodiment, the anti-lipolytic antibody can be any other antibody type (i.e., IgA, IgM, IgG, IgD, and IgE) obtained from an animal. In this embodiment, the anti-lipolytic antibodies, after administration to the ruminant, binds to the lipolytic immunogen, thereby protecting the unsaturated fatty acids and poly-unsaturated fatty acids from lipolysis, thereby increasing the amount of unsaturated fatty acids and poly-unsaturated fatty acids that pass through the rumen and which are absorbed into the ruminant's blood stream (compared to amounts of unsaturated fatty acids and poly-unsaturated fatty acids that pass through the rumen of ruminants that do not receive the anti-lipolytic antibody).

To generate anti-lipolytic antibodies, an immunogenic composition is administered to an animal in an amount effective to generate an immune response. To generate IgY anti-lipolytic antibodies, the immunogenic composition is administered to hens. The immunogenic composition contains at least one lipolytic immunogen, a pharmaceutically acceptable carrier, and optionally an adjuvant. The lipolytic immunogen can be lipase, another lipolytic enzyme, a lipolytic co-factor, at least one species of bacteria that produces one of these compounds (lipase, other lipolytic enzymes, or co-factors), or a combination thereof. The enzymes and co-factors can be purified or unpurified. The lipolytic immunogen can also be a fragment of lipase, another lipolytic enzyme, or lipolytic co-factor (i.e., a polypeptide) so long as when the anti-lipolytic antibody binds to the lipolytic immunogen, the lipolytic immunogen's activity is reduced. Lipase-producing bacteria include, but are not limited to, Propionibacterium acnes, Propionibacterium avidum, Anaerovibrio lipolyticus, and Butyrivibrio fibrisolvens. Lipase-producing bacteria also include other ruminal bacteria that produce lipase or are involved in the production of other lipolytic enzymes or lipolytic co-factors. In one embodiment, the immunogenic composition contains a combination of any of the above described lipolytic immunogens.

In another embodiment, the unsaturated fatty acid protectant is administered directly to a ruminant. This unsaturated fatty acid protectant is an immunogenic composition containing at least one lipolytic immunogen (discussed above), a pharmaceutically acceptable carrier, and optionally an adjuvant. This immunogenic composition is administered to the ruminant in an amount effective to produce an immune response in the ruminant. In one embodiment, the ruminant's immune response is the production of secretory antibodies that bind to the lipolytic immunogen. In another embodiment, the ruminant's immune response is the production of other types of antibodies. In these embodiments, the antibodies, when bound to the lipolytic immunogen, reduce the rate of lipolysis in the rumen (compared to lipolysis rates in the rumen of ruminants that do not produce the anti-lipolytic immunogen antibody) and thus protect the unsaturated fatty acids and poly-unsaturated fatty acids, thereby enhancing the absorption of unsaturated fatty acids and/or poly-unsaturated fatty acids into the blood stream (compared to amounts of unsaturated fatty acids and/or poly-unsaturated fatty acids that pass through the rumen of ruminants that do not receive the anti-lipolytic antibody).

A third invention described herein reduces the saturation of unsaturated fatty acids and poly-unsaturated fatty acids in the rumen of a ruminant by administering to the ruminant a biohydrogenating-inhibiting agent in an amount effective to reduce the saturation of the unsaturated fatty acids and poly-unsaturated fatty acids. The reduction in saturation occurs via reducing the enzymatic activity of saturase, lipase, other enzymes involved in biohydrogenation (biohydrogenation enzymes), and/or co-factors involved in biohydrogenation (biohydrogenation co-factors) This biohydrogenating inhibiting agent is, in one embodiment, an anti-saturation antibody which binds to an epitope on (i) saturase, (ii) bacterial saturase, (iii) lipase, (iv) bacterial lipase, (v) another enzyme(s) involved in ruminal biohydrogenation, (vi) a co-factor involved in ruminal biohydrogenation, and/or (vii) at least one ruminal bacterial species that produces a biohydrogenation enzyme and/or a biohydrogenation co-factor. One can administer the anti-saturation antibody to the ruminant orally, i.e., in animal feed or water or other composition that the ruminant ingests. In this manner, the anti-saturation antibody is swallowed and transported to the rumen. Of course, other modes of administration of this anti-saturation antibody are possible so long as the anti-saturation antibody reaches the rumen, interacts with its immunogen (a hydrogenation immunogen), and reduces the amount of biohydrogenation in the rumen. The anti-saturation antibody can be coated (contained within a protective shell) or uncoated. In one embodiment, the anti-saturation antibody are IgY antibodies obtained from hens. In another embodiment, the anti-saturation antibody is any other antibody type (i.e., IgA, IgM, IgG, IgD, and IgE) obtained from animals. In this embodiment, the anti-saturation antibody, after administration to the ruminant, binds to a hydrogenation immunogen and reduces the rate of saturation of the unsaturated bonds in the unsaturated fatty acids in the rumen (compared to rate of saturation in the rumen of ruminants that do not receive the anti-saturation antibody).

To generate anti-saturation antibody, an immunogenic composition is administered to an animal in an amount effective to generate an immune response. To generate IgY anti-saturation antibody, the immunogenic composition is administered to a hen in an amount effective to generate an immune response. The immunogenic composition contains at least one hydrogenation immunogen, a pharmaceutically acceptable carrier, and optionally an adjuvant. The hydrogenation immunogen can be (i) saturase, (ii) bacterial saturase, (iii) lipase, (iv) bacterial lipase, (y) another enzyme(s) involved in ruminal biohydrogenation, (vi) a co-factor involved in ruminal biohydrogenation, (vii) a fragment of a biohydrogenation enzyme or biohydrogenation co-factor, (viii) at least one ruminal bacterial species that produces a biohydrogenation enzyme and/or a biohydrogenation co-factor, and/or (ix) a combination thereof. The biohydrogenation enzymes and/or biohydrogenation co-factors can be purified or unpurified. The fragment of biohydrogenation enzyme or biohydrogenation co-factor are such that when the anti-saturation antibody binds to its epitope, the hydrogenation immunogen's activity is reduced. Saturase-producing bacteria include, but are not limited to, Butyrivibrio fibrisolvens and other ruminal bacteria that produce saturase or other biohydrogenation enzymes or co-factors. Lipase-producing bacteria include, hut are not limited to, Propionibacterium acnes, Propionibacterium avidum, and Anaerovibrio lipolyticus. Lipase-producing bacteria also include other ruminal bacteria that produce lipase or are involved in the production of other lipolytic enzymes or lipolytic co-factors. In one embodiment, the immunogenic composition contains a combination of any of the above described hydrogenation immunogens.

In another embodiment, the biohydrogenating-inhibiting agent is administered directly to a ruminant. In this embodiment, the biohydrogenating-inhibiting agent is an immunogenic composition containing at least one hydrogenation immunogen discussed above, a pharmaceutically acceptable carrier, and optionally an adjuvant. This immunogenic composition is administered to the ruminant in an amount effective to produce an immune response in the ruminant. In one embodiment, the ruminant's immune response is the production of secretory IgA antibodies that bind to the hydrogenation immunogen. In another embodiment, the ruminant's immune response is the production of other types of antibodies. In these embodiment, the antibodies, when bound to the hydrogenation immunogen, reduce the rate of biohydrogenation of unsaturated fatty acids in the rumen (compared to biohydrogenation rates in the rumen of ruminants that do not produce the anti-hydrogenation immunogen antibody).

An “immunogenic composition” is a composition that contains at least one antigen (also referred to as an “immunogen”) where administration of the immunogenic composition to an animal results in an immune response. In this invention, the immunogen can be lipase, fragments of lipase, saturase, fragments of lipase, ruminal bacteria, and/or other enzymes and/or co-factors, all of which are involved in lipolysis and/or biohydrogenation of unsaturated fatty acids in the rumen of a ruminant. The ruminal bacteria can be live, attenuated; live and not attenuated; inactivated; membrane fractions derived from the ruminal bacteria; and/or bacterial ghosts of the ruminal bacteria. A bacterial ghost is bacterium containing a pore in its cell wall and/or cell membrane and through which the cytosol exits the bacterium, thus killing the bacterium. However, the bacterium's morphology (surface proteins and other structures s preserved. See, Langemann, et al., Bioengineered Bug 1(5)326-336 (2010). The lipase, saturase, and fragments thereof can be unpurified; contained in culture media; or in the presence of other compounds (proteins, lipids, sugars, etc.). The fragments of saturase and/or lipase do not have be contiguous amino acids to be an effective immunogen. A fragment of saturase and/or lipase should generate an antibody that binds to the fragment and causes a reduction in activity of the enzyme to which it is bound. Additional antigens can also be included in the immunogenic compositions, if one seeks to generate antibodies to those other antigens even if those antigens are not involved in lipolysis or biohydrogenation in ruminants. The immunogenic composition can be combined with animal feed or water or administered to the animal via other routes.

An “immunological response” or “immune response” to an immunogen or immunogenic composition is, in an animal, the development, increase, or decrease of a humoral and/or a cellular immune response to the immunogen, whether presented by itself or presented in the immunogenic composition. The immune response may be an increased or enhanced immune response (immuno-stimulatory) or a decrease or suppression of an immune response (immuno-suppressant). The immune response may be a systemic and/or localized immune response. A “humoral immune response” refers to an immune response mediated by antibody molecules, while a “cellular immune response” is one mediated by T-lymphocytes and/or other white blood cells. In one embodiment for this invention, the immune response is humoral, in that the hen lays eggs containing the desired IgY. In another embodiment for this invention, the immune response is humoral, in that the ruminant generates IgA which is secreted into the saliva (and swallowed so that the enter the rumen) or other types of antibodies.

An antibody (also referred to as immunoglobulin) is a protein that contains an antigen binding site or paratope for a specific molecule. Antibodies are produced by B lymphocytes in an animal after the animal is exposed to a protein, compound, organism, etc. (referred to an antigen or immunogen). Antibodies are part of the humoral immune response in an animal. The antibody may be a member of any immunoglobulin class, including IgY, IgG, IgM, IgA, IgD, and IgE. In one embodiment of this invention, IgY antibodies are used. In another embodiment of this invention IgA antibodies are used. IgA antibodies are secretory antibodies and are present in saliva and other mucosal tissue. “Antibody” or “antibodies” encompass polyclonal and monoclonal antibody preparations, as well as preparations including hybrid antibodies, altered antibodies, chimeric antibodies and, humanized antibodies, as well as: hybrid (chimeric) antibody molecules (see, for example, Winter et al., Nature 349:293-299 (1991); and U.S. Pat. No. 4,816,567); F(ab′)₂ and F(ab) fragments; Fv molecules (noncovalent heterodimers, see, for example, Inbar et al., Proc. Natl. Acad. Sci. USA 69:2659-2662 (1972); and Ehrlich et al., Biochem. 19:4091-4096 (1980)); single-chain Fv molecules (sFv) (see, (.g., Huston et al., Proc. Natl. Acad Sci. USA 85:5879-5883 (1988)); dimeric and trimeric antibody fragment constructs; minibodies (see, e.g., Pack et al., Biochem. 31:1579-1584 (1992); Cumber et al., J. Immunology 149B:120-126 (1992)); humanized antibody molecules (see, e.g., Riechmann et al., Nature 332:323-327 (1988); Verhoeyan et al., Science 239:1534-1536 (1988); and U.K. Patent Publication No. GB 2,276,169, published 21 Sep. 1994); and any functional fragments obtained from such molecules, wherein such fragments retain specific-binding properties of the parent antibody molecule.

“Vaccination”, “vaccinate”, “immunization”, “immunize”, and “inoculate” are synonymous and are the administration of the immunogen or the immunogenic composition. Immunization can also include removing immunological cells from the animal, allowing such immunological cells to interact with an antigen in-vitro, and then returning those immunological cells or their progeny back to the animal's body. Exemplary routes of administration of an immunogen or immunogenic composition of this invention include, but not limited to, intramuscular injection, intraperitoneal injection, subdermal injection, intradermal injection, subcutaneous injection, intravenous injection, oral administration, sublingual administration, vaginal administration, rectal administration, transmucosally, transcutaneous adsorption, intranodal administration, intracoronary administration, intraarterial administration, intratracheal administration, intraarticular administration, intraventricular administration, intracranial administration, intraspinal administration, intraocular administration, aural administration, inhalation, and intranasal administration. For this invention, oral administration may involve an animal eating a plant or plant cells which contain the antigen, eating a mixture of animal feed and the immunogenic composition, or drinking a mixture of a liquid and the immunogenic composition. The immunogenic composition can be mixed together with the feed, sprinkled onto the animal feed, or coating the animal feed. Any suitable liquid (e.g., water) can be used. Such oral administration may result in mucosal immunity. In one embodiment, to generate an secretory IgA response in a ruminant, one administers the immunogenic composition nasally, orally, sublingually, vaginally, rectally, and/or transmucosally via any other mucosal tissue in the animal.

The immunogenic compositions of this invention may contain one or more pharmaceutically acceptable carriers. Non-limited examples of such carriers include phosphate buffered saline (PBS), lactose, dextrose, sucrose, sorbitol, mannitol, starch, acacia gum, calcium phosphate, calcium silicate, water, syrup, methyl hydroxybenzoate, propyl hydroxybenzoate, talc, stearic acid, magnesium, mineral oil, and polymers such as alginate, gelatin, microcrystalline cellulose, methyl cellulose, cellulose, polyvinylpyrrolidone, poly-lactic acid (PLA), poly-glycolic acid (PGA), and poly-lactic-co-glycolic acid (PLGA). In addition to the above carriers, the immunogenic compositions of the present invention may further comprise lubricants, wetting agents, sweetening agents, flavoring agents, emulsifiers, suspending agents, preservatives, etc. Suitable pharmaceutically acceptable carriers and formulations are described in detail in Remington's Pharmaceutical Sciences (19th ed., 1995). While the immunogenic compositions of the present invention are used in animals, but humans, “pharmaceutically acceptable” refers to those items, compounds, etc. that are approved for use in human and/or in animals.

The immunogenic compositions of this invention may optionally contain one or more adjuvants. Non-limiting examples of adjuvants include mineral oil, vegetable oil, water-in-oil emulsion, oil-in-water emulsion, water-in-oil-in-water emulsion, aluminum hydroxide, aluminum phosphate, squalene and squalene-like compounds, Freund's complete adjuvant, Freund's incomplete adjuvant, muramyl dipeptide, monophosphoryl lipid A, polyphosphazine, E. coli LT (or LT-B, native or toxoid), Vibrio cholera toxin (CTX or CT), CpG motif containing oligonucleotide, and compounds that interact with Toll-like Receptors (TLR).

The appropriate dose of the immunogenic composition of the present invention depends on several variables such as the formulation, the route of administration, the animal's age, the animal's weight, the time of administration, the excretion rate, and reaction irritability. One of ordinary skill in the art can determine the appropriate dose by administering the antigen to the animal and assaying for an increase or, if applicable, a decrease in the immune response. The animal could be provided one, two, three, four or more doses that are administered one or two or three or more weeks apart from each other. The animal could also be inoculated yearly or every other year or with less frequency with the number of inoculations as previously described. Alternatively, the animal could be administered the immunogenic composition more frequently.

The expressions “effective dosage”, “effective amount”, “amount effective to”, and similar phrases are that amount which will induce the production of antibodies (humoral immune response) and/or cell-mediated immune response in an animal against the immunogen contained in the immunogenic composition. Immunity is considered as having been induced in the animal when the humoral and/or cell-mediated immune response to the immunogen is significantly higher than the humoral and/or cell-mediated immune response in an unvaccinated control group. The presence of IgY in eggs and the presence of IgA in ruminants indicate that the hen and ruminant, respectively, receiving an effective amount of the immunogenic composition. The actual effective amount or effective dose of the immunogenic composition may vary depending on (i) the formulation, (ii) the antigen, (iii) the type of immunogenic composition (e.g., protein vaccine or DNA vaccine), (iv) the age of the animal. (v) the size/weight of the animal. (vi) the route of administration, (vii) the time of administration, (viii) the excretion rate, and (ix) the reaction irritability. One of ordinary skill in the art can determine the appropriate dose by administering the immunogenic composition to the ungulate and assaying for an increase or, if applicable, a decrease in the immune response. An antigen dose response assay is such an assay for assess the immune response.

In one embodiment, the suitable dosage of the immunogenic composition described herein can range from approximately 10 ng/kg/day to approximately 1 g/kg/day of the body weight of the animal receiving the immunogenic composition. In another embodiment, the suitable dosage of the immunogenic composition described herein can range from approximately 100 ng/kg/day to approximately 100 mg/kg/day of the body weight of the animal receiving the immunogenic composition. In yet another embodiment, the suitable dosage can range from approximately 100 ng/kg/day to approximately 100 ng/kg/day of the body weight of the animal receiving the immunogenic composition. In yet another embodiment, the dosage can be approximately 500 ng/kg/day of the body weight of the animal receiving the immunogenic composition.

In one embodiment, the antibody that is administer to the ruminant is administered one, two, three, or four or more times per day. In another embodiment, the antibody is administered every one, two, three, four, or more days. The amount of antibody that is administered depends on the number of times that the antibody is provided to the animal. The amount may also depend on the specificity of the antibody to the immunogen. In one embodiment, one administers between approximately 100 ng antibody/kg body weight/day to approximately 500 mg antibody/kg body weight/day. In another embodiment, one administers between approximately 1 μg/kg body weight/day to approximately 50 mg antibody/kg body weight/day. In yet another embodiment, one administered between approximately 10 μg/kg body weight/day to approximately 1 mg antibody/kg body weight/day. In yet another embodiment, one administered between approximately 20 μg/kg body weight/day to approximately to 200 μg/kg body weight/day. In one embodiment, one administers purified antibody to the animal. In another embodiment, the antibody is not purified from the other components of the egg. In one embodiment, the egg can be mixed with animal feed. In another embodiment, the antibody is mixed with a liquid.

In one embodiment, the immunogenic composition that are administered to hens or ruminants contains one or more species of ruminal bacteria. The amount of each bacteria species present in each dose of the immunogenic composition can range from between approximately 10³ CFU/kg body weight to approximately 10¹¹ CFU/kg body weight in one embodiment. In another embodiment, the amount of each bacteria species can range between approximately 10⁵ CFU/kg body weight to approximately 5×10⁹ CFU/kg body weight. In another embodiment, the amount of each bacteria species can range between approximately 10⁷ CFU/kg body weight to approximately 10⁹ CFU/kg body weight.

The amount of lipase and/or saturase or fragments thereof in the immunogenic composition can range between approximately 100 ng/kg body weight to approximately 1 g/kg body weight in one embodiment. In another embodiment, the amount of each protein or polypeptide in the immunogenic composition can range from approximately 1 μg/kg body weight to approximately 100 mg/kg body weight. In another embodiment, the amount of each protein or polypeptide in the immunogenic composition can range from 10 μg/kg body weight to approximately 10 mg/kg body weight.

As discussed above, the antibodies being administered to a ruminant (purified or unpurified) can be coated with an edible coating. Any edible coating can be used as long as it does not negatively affect the function of the vaccine. Examples of useful coatings in the present invention include methylmethacrylates including, for example, Eudragit® (Rohm and Hass) and Kollicoat® (BASF); zein, cellulose derivatives (e.g., cellulose acetate, cellulose phthalate, ethylcellulose, and hydroxylpropylmethylcellulose), fats and waxes.

The invention also includes kits containing one or more containers of the immunogenic compositions and/or immunogens of the invention. The immunogenic composition can be in liquid form or can be lyophilized; as can be the immunogens. Suitable containers for the immunogenic compositions and/or immunogens include, for example, bottles, jugs, vials, syringes, and test tubes. Containers can be formed from a variety of materials, including glass or plastic. A container may have a sterile access port (for example, the container may be an intravenous solution bag or a vial having a stopper pierceable by a hypodermic injection needle). The kit can also contain a second container inside of which is a pharmaceutically acceptable carrier, such as phosphate buffered saline (PBS), Ringer's solution, or dextrose solution. The kit can also contain other materials useful to the end-user, including other pharmaceutically acceptable formulating solutions such as carriers, filters, needles, and syringes or other delivery devices. The kit may optionally include an adjuvant in a container. The kit can also contain written instructions for administering the immunogenic composition and/or immunogen and other contents of the kits to animals. The written instructions may describe methods for inducing an immune reaction or methods for treating infections. The invention also includes a delivery device pre-filled with the immunogenic composition of the invention.

In another embodiment, the kit could contain purified IgY or egg yolk or the entire contents of an egg in liquid or lyophilized form in one container. The kit could also contain a carrier in another container. The carrier can be mixed with the IgY, egg yolk, or entire egg, prior to administration to the ruminant or adding to the ruminant's feed or water.

The terms “approximately” and “about” refer to a quantity, level, value or amount that varies by as much as 30%, or in another embodiment by as much as 20%, and in a third embodiment by as much as 10% to a reference quantity, level, value or amount. As used herein, the singular form “a”, “an”, and “the” include plural references unless the context clearly dictates otherwise. For example, the term “a bacterium” includes both a single bacterium and a plurality of bacteria.

Having described the invention in general terms, below are examples illustrating the generation and efficacy of the invention. Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation. All documents cited herein are incorporated by reference.

### EXAMPLE 1

### Antibody Development

Anaerovibrio lipolyticus, Butyrivibrio fibrisolvens strain H17C, Propionibacterium avidum, and Propionibacterium acnes are prominent ruminal bacteria that have been identified as contributors to lipolysis in the rumen. To test this technology in vitro, chicken IgY antibodies are generated against these bacteria, isolated from the egg yolks and are tested as inhibitors of bacterial lipolytic activity. Additionally, an antibody generated from chicken egg yolks against a Pseudomonas lipase is tested to determine if an antibody raised against the purified protein would be more effective than antibodies raised against whole cell preparations.

To develop the IgY antibodies, separate 20 ml early stationary phase cultures of each of the lipase-producing bacteria containing approximate 10e8 to 10e9 colony forming units are separately centrifuged for 5 minutes at 4000×g. The resultant supernatant fluid from each bacterium is poured off, and each pellet is resuspended individually in 2 ml 0.1 M sodium phosphate buffered saline (PBS) (pH 7). Each solution is re-centrifuged for 5 minutes at 4000×g, and the supernatant is poured off. This pellet is resuspended individually in 2 ml PBS (pH 7). The centrifuging the bacteria and resuspending the pellet is again repeated a second time for a total of two washes. Following the final wash, each individual bacterial cell preparation is suspended in separate containers with a combination of 1 mL of PBS and 1 mL of Adjuvant Titer Gold Max (Sigma Aldrich, St. Louis, Mo.). Each separate suspension is mixed and then injected into its own set of laying hens (0.6 ml of each suspension injected 0.1 ml at a time into 6 different locations in the muscle). At 3 weeks and 6 weeks after the first inoculation, 1 mL PBS of each bacterial suspension is mixed with 1 ml normal saline and then injected into its set of laying hens (0.6 ml of each suspension injected 0.1 ml at a time into 6 different locations in the muscle). After the final injection, at least 1 week is allowed to pass before collection of eggs and purification of the anti-whole cell IgY antibodies. Purification of IgY antibodies is accomplished using an Eggcellent IgY purification kit (Thermo Scientific Pierce, Rockford, Ill.).

Between 2 mg and 3 mg purified P. acnes lipase or the commercially available Pseudomonas lipase (Sigma Aldrich, St. Louis, Mo.) are prepared with 1 ml Adjuvant Titer Gold Max (Sigma Aldrich, St. Louis, Mo.) or 1 ml PBS as described above and are inoculated in chickens as described above for the whole bacterial cells. After the final injection, at least 1 week is allowed to pass before collection of eggs and purification of the anti-whole cell IgY antibodies. Purification of IgY antibodies is accomplished using an Eggcellent IgY purification kit (Thermo Scientific Pierce, Rockford, Ill.).

### EXAMPLE 2

### In-Vitro Assay of Chicken IgY

To test the effectiveness of the antibodies generated in Example 1 above, each bacterium is cultured under anaerobic conditions in a standard broth medium which contained (per I.): 292 mg K2HPO₄, 292 mg KHPO₄, 480 mg (NH₄)₂SO₄, 480 mg NaCl, 100 mg MgSO₄.7H₂O, 64 mg CaCl₂.2H₂O, 4,000 mg Na₂CO₃, 600 mg cysteine HCl, 10 g trypticase (BBL Microbiology Systems, Cockeysville, Md.), 2.5 g yeast extract, branched-chain fatty acids (1 mmol each of isobutyrate, isovalerate, and 2-methylbutyrate), hemin, vitamin mix (20 mg each thiamine, pantothenate, nicotinamide, pryridoxine HCl, riboflavin, 1 mg p-aminobenzoic acid, 0.5 mg biotin, 0.5 mg folic acid, 0.2 mg vitamin B-12, and 0.5 mg lipoic acid) trace minerals (Cotta and Russel, J. Dairy Sci. 65:226-234 (1982)) and 0.02% (wt/vol) glucose. The medium is further prepared by boiling to remove dissolved O₂ and then saturated with O₂-free gas while cooled on ice under a continuous flow of 100% CO₂The cooled medium is distributed (6 mL/tube) using the anaerobic Hungate technique as described by Bryant (Am. J. Clin. Nutr. 25:1324-1328 (1972)) to 18×150 mm glass tubes which are pre-loaded with 0.2 mL olive oil. Tubes are then autoclaved and inoculated with the respective culture. Cultures are grown until late log phase for 24-96 hours. Late log phase for each culture is determined using a spectrophotometer to measure absorbance at 62.0 wavelength. The following absorbance are measured to be late log phase for each culture: A. lipolyticus-abs 1.0-1.2, B. fibrisolvens H17C-abs 0.4-0.5, P. acnes-abs 1.1-1.2, and P. avidum-abs 1.2-1.3. Once late log phase is reached for each culture, 4 mL of culture is then transferred to tubes containing 2 mL anaerobic assay buffer. The anaerobic assay buffer is composed of the same concentration of minerals and cysteine HCl as in the standard broth medium but lacks all other ingredients and is prepared using the same anaerobic methods. Tubes containing the buffer also contain 0.3 mL olive oil and abed of glass beads. Tubes are treated without (negative controls) or with increasing amounts of chicken IgY antibody provided in Example 1 (0.2 mg, 1.0 mg, 2.5 mg, and 5.0 mg). Each antibody is tested against each bacterium to determine if any cross-reactivity exists among the antibodies. Tubes are incubated for 12 hours at 39° C. Results indicate that all five antibodies, at each dose are effective at reducing lipolytic activity against each bacterium with the anti-Pseudomonas lipase being the most effective, averaging almost a 70% reduction in lipolytic activity across all bacteria at the highest antibody dose (5.0 mg).

Butyrivibrio fibrisolvens uniquely participates in both lipolysis and biohydrogenation processes, and thus the anti-B. fibrisolvens strain H17C antibodies generated in Example 1 are also tested to determine if the antibodies are effective at reducing biohydrogenation against a pure culture of B. fibrisolvens strain H17C. To test the antibody's potential inhibitory effects on biohydrogenation, tubes are treated without and with 0.6 mL of the anti-B. fibrisolvens strain H17C antibodies. Tubes are supplemented with 6 mg of either linoleic (18:2n-6) or α-linolenic acid (18:3n-2) and are incubated for 1 hour and 3 hours at 39° C. Biohydrogenation of α-linolenic acid is depressed by 45% compared to negative controls. This example demonstrates that ruminal bacteria responsible for freeing and hydrogenating ruminal fatty acids can be immunologically inhibited in vitro.

In a subsequent study, the chicken IgY produced above in Example 1 are tested for their ability to inhibit lipolytic activity of mixed cultures of rumen microbes. Ruminal fluid is collected from a cannulated cow, and 6 mL are distributed anaerobically into tubes containing 21 g of glass beads and 0.3 mL olive oil to measure lipolytic activity, or 0.3 mL of linseed oil or corn oil to examine biohydrogenation. Tubes containing olive oil are treated without (negative control) or individually with increasing doses of each antibody (0.2 mg, 1.0 mg, 2.5 mg, and 5.0 mg) and are incubated for 12 hour at 39° C. Tubes containing either linseed oil or corn oil receive 0.6 mL of the anti-B. fibrisolvens strain H17C antibodies and are analyzed for biohydrogenation products after 0 hour, 3 hour, and 12 hour incubation. To measure biohydrogenation products, total lipids are extracted from the tubes by the protocol described in Folch, et al. (J. Biol. Chem. 226:497-509 (1957)). The lipids are then methylated according to protocol reported by (Morrison and Smith (J. Lipid Res. 5:600-608 (1964)). The fatty acid methyl esters (FAME) are then analyzed using a gas chromatograph (Agilent Technologies, Inc., Santa Clara, Calif.) using the method described by Sturdivant, et al. (Meat Sci. 32:449-458 (1992)), Lipolytic activity is measured by the rate of free fatty acids that are liberated using the colorimetric assay described by Kwon and Rhee (J. Am. Oil Chem. Soc. 63:89-92 (1986)).

The anti-A. lipolyticus 5s, B. fibrisolvens H17C, P. acnes, and P. avidum antibodies all show a percent reduction in accumulation of FFA when introduced against mixed culture at every dose administered. Percent reduction in FFA does not differ (P>0.05) amongst each dose for any of the antibodies. The antibodies against A. lipolyticus 5s result in the greatest overall decrease in percent lipolytic activity from negative control samples, 34.5±4.1% (mean±SEM). respectively. In cultures incubated for 12 hours with linseed oil, total trans-fatty acid isomers decrease from 17.0±0.69 μmol in negative control samples to 14.2±0.69 μmol in antibody-treated cultures (time×treatment P<0.0568). There is no measurable lipolytic or hydrogenation activity in mixed cultures incubated with corn oil. These results are consistent with previously published study demonstrating hat linseed oil is more readily hydrolyzed than corn oil (see, Edwards, et al. (2013)).

### EXAMPLE 3

### Production of Recombinant P. Acnes Lipase and IgY Against the Recombinant P. Acnes Lipase

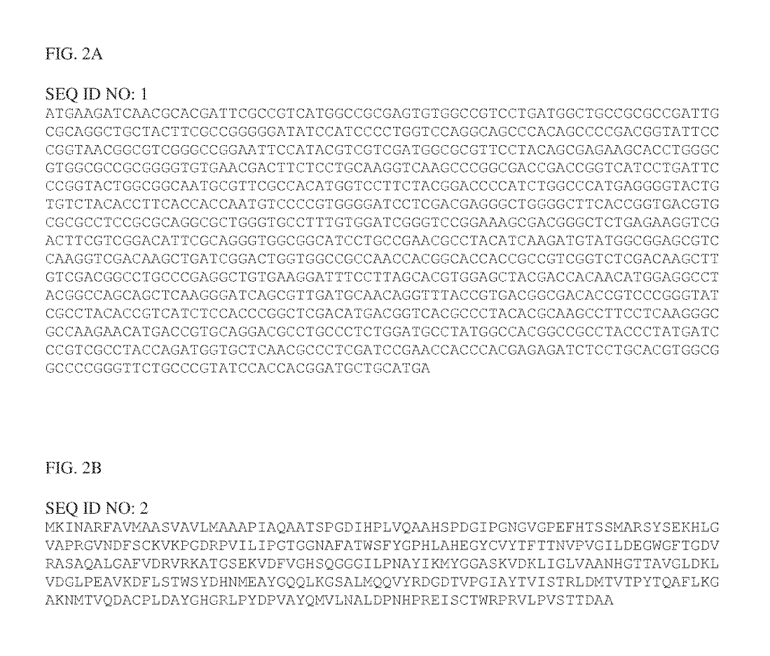

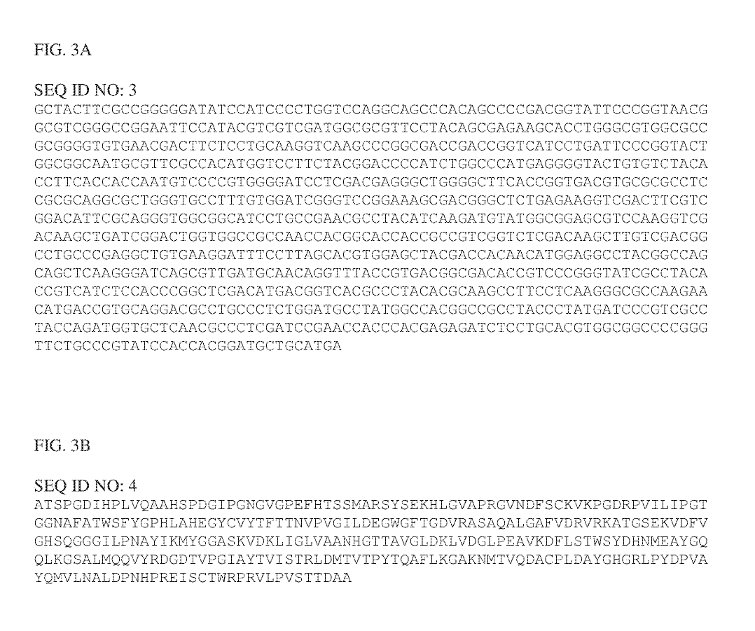

In an attempt to generate antibodies with stronger inhibitor activity against lipase, recombinant lipase is prepared. Propionibacterium acnes lipase is encoded in gheA. It is 339 amino acids long as a pro-protein (containing a secretion signal peptide) and 313 amino acids long as a mature protein. See FIG. 2A and FIG. 2.B for the DNA sequence (SEQ ID NO: 1) and amino acid sequence (SEQ ID NO: 2), respectively, for pro-lipase (gheA). See FIG. 3A and FIG. 3B for the DNA sequence (SEQ ID NO: 3) and amino acid sequence (SEQ ID NO: 4), respectively, for mature lipase. First, a portion of P. acnes gheA encoding amino acids 27-339 (the mature protein without the secretion signal peptide) is cloned by PCR using the following primers: gheA-F2: CGCGAACAGATTGGAGGTGCTACTTCGCCGGGGGATATC (forward primer, SEQ ID NO: 5); and gheA-R: GTGGCGGCCGCTCTATTATGCAGCATCCGIGGTGGATAC (reverse primer, SEQ ID NO: 6). A crude template of P. acnes genomic DNA is prepared by lysing P. acnes cells with QuickExtract DNA Extraction Solution (Epicentre Technologies, Madison, Wis.) according to the manufacturer's protocol. Reactions (50 μl) contain 2.5 pmol of each primer and 5 μl genomic DNA template in 1× AmpliTaq Gold PCR Master Mix (Applied Biosystems, Foster City, Calif.). Reactions are subjected to 5 minute incubation at 95° C. followed by 30 cycles of PCR with a denaturation temperature of 95° C. (0.5 minute), an annealing temperature of 50° C. (0.5 minute), and an extension temperature of 72° C. (1 minute). The resulting 975 base pair amplicon is cloned into plasmid pRham N-His SUMO Kan using the Expresso Rhamnose SUMO Cloning and Expression system (Lucigen Corp., Middleton, Wis.) according to the manufacturer's instructions. The resulting plasmid is named pRham-gheA.1.

Plasmid pRham-gheA. 1 is transformed into E. cloli® 10G chemically competent cells (Lucigen Corp., Middleton, Wis.) according to the manufacturer's instructions. Two Fernbach flasks, each containing one liter of Luria-Bertani (LB) broth supplemented with kanamycin (30 μg/ml), D-glucose (0.15% (w/v)) and L-rhamnose (0.2% (w/v)), are each inoculated with 10 ml of an overnight culture of E. cloni 10G/pRham-gheA.1. The cultures are incubated at 37° C. with shaking (200 rpm) overnight (20 hours), and the cells are harvested by centrifugation (5000×g, 15 minutes).

Cells are lysed by re-suspending in 60 ml B-PER. Bacterial Protein Extraction Reagent (Thermo Scientific, Rockford, Ill.) plus DNaseI (10 U/ml) and RNaseA (8 U/ml). Following 30 minute incubation at room temperature, the insoluble material (containing the bulk of the recombinant GheA protein) is collected by centrifugation (10,000×g, 15 minutes). The insoluble protein is solubilized by re-suspending the pellet in 60 ml of 8 M urea in phosphate buffered saline (140 mM NaCl, 12 mM sodium phosphate, pH 7.4 (PBS-7.4)), followed by centrifugation (15,000×g, 15 minutes) to clear remaining insoluble material.

The supernatant liquid is diluted 1:10 in PBS-7.4. HisPur Ni-NTA Superflow Agarose (10 ml of 50% slurry; Thermo Scientific, Rockford, Ill.) is added, and the slurry is gently rocked for 30 minutes at room temperature. The resin is collected in a glass chromatography column, and is washed with 50 ml of 20 mM imidazole in PBS-7.4. Protein is eluted from the resin by washing with 30 ml of 250 mM imidazole in PBS-7.4. Purified protein (15 ml) is dialyzed against 2 L PBS-7.4.

To cleave the SUMO tag from the amino terminus of the recombinant GheA, dithiothreitol (2 mM) and 10 μl of SUMO-Express protease (1 U/μl; Lucigen Corp., Middleton, Wis.) are added to the retentate, and the mixture is incubated at 30° C. for 1 hour. HisPur Ni-NTA Superflow Agarose (1 ml of 50% slurry) and imidazole (10 mM) are added to the solution to bind the cleaved SUMO tag. The solution is cleared of Ni-NTA agarose by filtration through a chromatography column. The filtrate contains the recombinant GheA (mature lipase).

### EXAMPLE 4

### Cows Administered Chicken Antibodies Orally have Reduced Biohydrogenation and Bacterial Lipase Activity

Purified lipase from Example 3 is injected into hens using the protocol described in Example 1. Eggs are collected, and anti-P. acnes lipase IgY antibodies are obtained via purification as described in Example 1 above with concentration ranging from 7.4 mg/m1 to 11.5 Anti-B. fibrisolvens strain H17C IgY antibodies produced in Example 1 above are also purified to a concentration ranging from 7.4 mg/ml to 11.5 mg/ml. Both types of IgY are used in the first trial in this example.

To test oral administration of the IgY antibodies, a cross-over design trial is conducted with 8 lactating cows on an appropriate lipid-supplemented total mixed ration (TMR). Four animals are orally treated with the anti-P. acnes lipase/anti-B. fibrisolvens strain H17C IgY antibodies and then receive no treatment during the second period which occurs after a 21 day reacclimation period. Four animals receive no treatment during the first period and are then fed antibodies during a second period to be conducted after a 21 day reacclimation period. Treatments (a total of 17 g anti-P. acnes lipase IgY and an equal portion of anti-B. fibrisolvens strain H17C IgY) are administered twice daily for 2 days via oral drench. Blood samples are collected morning and night from the jugular vein beginning 2 days before oral IgY administration, during the 2 days of oral IgY administration, and for 2 days after completion of oral IgY administration. Milk is collected morning and night daily during the same time period. This design allows for observation of differences in concentrations of unsaturated fatty acid in blood and milk in each animal before and during treatment and even a possible diminishing effect in unsaturated fatty acid concentrations as each animal completed treatment. Moreover, this study allows for comparisons between non-treated and treated groups of animals.

A second study is conducted using a similar cross-over design trial with 8 lactating cows as described above, except that the cows are fed 17 g of each IgY against A. lipolyticus, P. acnes, P. avidum, and B. fibrisolvens strain H17C (all these IgY produced in Example 1). A 21 day reacclimation period occurs between the two test periods. Milk is collected morning and night daily during the same time period. This design allows for observation of differences in concentrations of unsaturated fatty acid in blood and milk in each animal before and during treatment and even a possible diminishing effect in unsaturated fatty acid concentrations as each animal completed treatment. Moreover, this study allows for comparisons between non-treated and treated groups of animals.

### EXAMPLE 5

### Ruminant Antibodies Reduce Biohydrogenation and Bacterial Lipase Activity

To examine the inhibitory effect of ruminant-produced antibodies in vivo, B. fibrisolvens strain H17C and purified P. acnes lipase (see Example 3 above) are purified and injected intra-muscularly into milking goats. The adjuvant, muramyl dipeptide, is mixed with the bacteria and/or lipase to promote the production of secretory IgA antibodies against the bacteria and/or lipase in the saliva of the goats. Because the IgA are present in the goat's saliva, the antibodies have direct contact with lipids prior to entering the rumen. Additionally, the ruminant's continuous production, secretion and swallowing of saliva provide continuous replenishment of secretory IgA to the rumen.

B. fibrisolvens strain H17C are used because of the bacterium's dual function in the rumen. To be effective against biohydrogenation as well as against lipolysis, attenuated whole cell bacterial preparation is injected into goats intra-muscularly, because, unlike lipolysis, biohydrogenation is an intracellular process. As shown in Example 2 above, in-vitro work demonstrates that IgY against a purified lipase is more effective in reducing lipolysis than IgY against whole bacterial cell preparation. Because of this result, P. acnes lipase is included in the immunogenic composition in addition to whole cell B. fibrisolvens H17C, because this organism has shown some of the high rates of lipolytic activity, and because, also shown in Example 2, each antibody is effective against a range of different bacterium.

Eight milking goats are injected intra-muscularly with a 3 mL immunogenic composition containing 300 μg P. acnes lipase, 300 μg whole cell B. fibrisolvens, 300 μg muramyl dipeptide, and sufficient saline to bring the volume to 3 mL. The 3 mL immunogenic composition is injected in 0.5 mL increments across six different injection sites in the goat's neck. A second immunogenic composition containing 300 μg P. acnes lipase, 300 μg whole cell B. fibrisolvens, and sufficient saline to bring the volume to 3 mL is administered intra-muscularly across six different injection sites in the goat's neck, 21 days after the initial injection. This second dosage acts as a booster for antibody production. Eight goats are not immunized to serve as the negative control. Milk samples are taken on days −6, −3, and 0 pre-treatment and on day 21 until day 63 after the first immunization on a weekly basis. Samples of ruminal fluid, collected by stomach tube, and blood, collected from jugular into vacuum tubes, are obtained on day 0 pre-treatment and on day 42 and day 63 after the first immunization.

Lipolytic rates by the ruminal fluid are tested in triplicate by pipetting 6 mL of the collected fluid into glass tubes containing 21 g glass beads and 0.3 mL olive oil. Following 12 hour incubation at 39° C., lipolytic activity is measured by the rate of free fatty acids that are liberated using the colorimetric assay described by Kwon and Rhee (J. Am. Oil Chem. Soc., 63:89-92 (1986)).

Volatile fatty acids (VFA) are also measured by retaining 1 mL of rumen fluid from each goat and mixing it with phosphoric acid and pivalic acid solution (1.0% w/v) as an internal standard. The mixed solution is then used to determine the concentration and composition of VFA using a gas chromatograph (Agilent Technologies, Inc., Santa Clara, Calif.) equipped with a flame ionization detector (HD) (Agilent Technologies, Inc., Santa Clara, Calif.) using the protocol described in Li, et al. (Asian-Aust. J. Anim. Sci. 22:1521-1530 (2009)). To measure changes in fatty acid profile, total lipids are extracted from blood and milk samples by the protocol described in Folch, et al. (J. Biol. Chem. 226:497-509 (1957)). The lipids are then methylated according to protocol reported by Morrison and Smith (J. Lipid Res. 5:600-608 (1964)). The fatty acid methyl esters (FAME) are then analyzed using a gas chromatograph (Agilent Technologies, Inc., Santa Clara, Calif.) using the method described by Sturdivant, et al. (Meat Sci. 32:449-458 (1992)).

To determine the overall effect the immunogenic composition has on lipolytic activity, fatty acid profiles, and volatile fatty acids, a co-variant general ANOVA (Statistix v.9.0, Analytical Software, Tallahassee, Fla.) with repeated measure and an LSD separation of means (P<0.10) are performed on the results. The model includes two main effects, immunogenic composition treatment and period of sample collection. Results pre-treatment serve as the co-variant and the period of sampling represents the repeated measure.

The lipolytic activity from incubated rumen fluid from vaccinated and negative control goats is shown in Table 1. Period 1 is day 42 and Period 2 is day 63 after the first immunization. Results do not show a significant treatment effect for rumen fluid rates, however, at both periods the rates are numerically lower for the vaccinated goats. A significant period effect (P<010) is observed showing an increase in lipolytic rates over time.

TABLE 1

| nmol FFA/mL/hr¹ | | | | | | | |
|---|---|---|---|---|---|---|---|
| Period 1 | | Period 2 | | | | | |
| Neg. control | Treated | Neg. control | Treated | SE | Treatment | Period | Interaction |
| 543.89 | 487.59 | 919.53 | 884.32 | 106.36 | 0.7918 | 0.0027* | 0.9224 |

¹Values are the mean from n = 8 animals
*Means differ (P < 0.10)

Fatty acid profiles of milk are analyzed every week starting on day 21 following the first immunization. For the milk analysis. Period 1 is the average 3 analysis from each week beginning on day 21 after the first immunization to day 42. Period 2 is the average of 3 analysis from each week beginning on day 49 after the first immunization to day 63. The fatty acid profile of blood is analyzed on day 42 post-first immunization representing Period 1 and day 63 post-first immunization representing Period 2. Table 2 shows the saturated fatty acid lipid profile for both blood and milk. Results show that antibodies generated by the goats increase fatty acid C10:0 in milk at the longest time period post-immunization. Similarly, a significant treatment effect for fatty acid C22:0 exists, demonstrating an increase in this fatty acid after immunization. As seen in Table 2, there is a significant interaction for fatty acid C 16:0 in the blood demonstrating a decrease over time after immunization. Similarly, fatty acid C16:0 also appears to be numerically lower in the milk of animals that are immunized. Although not significant, at both periods, total saturated fatty acids demonstrate a reduction trend in milk from animals receiving the immunogenic composition.

TABLE 2

| | Area % of Total Lipid¹ | | | | | | | |
|---|---|---|---|---|---|---|---|---|
| | Period 1 | | Period 2 | | | | | |
| Fatty | Neg. | | Neg. | | | | | |
| Acid | control | Treated | control | Treated | SE | Treatment | Period | Interaction |

Milk

| | | | | | | | | |
|---|---|---|---|---|---|---|---|---|
| C10:0 | 4.4249 | 3.8380 | 4.1767 | 4.2848 | 0.1749 | 0.5060 | 0.5792 | 0.0669* |
| C12:0 | 3.4235 | 2.8678 | 3.8399 | 3.1941 | 0.1466 | 0.1615 | 0.0239* | 0.7632 |
| C14:0 | 10.302 | 9.770 | 10.813 | 10.201 | 0.2548 | 0.3293 | 0.0859* | 0.8767 |
| C16:0 | 30.994 | 30.425 | 30.595 | 30.365 | 0.4827 | 0.8322 | 0.6418 | 0.7312 |
| C17:0 | 0.5471 | 0.5360 | 0.5598 | 0.5482 | 0.0117 | 0.5587 | 0.3070 | 0.9849 |
| C18:0 | 11.545 | 12.062 | 11.093 | 12.012 | 0.3330 | 0.5157 | 0.4637 | 0.5552 |
| C20:0 | 1.4331 | 1.5423 | 1.4220 | 1.4579 | 0.0619 | 0.5784 | 0.4534 | 0.5633 |
| C22:0 | 0.0422 | 0.0508 | 0.0385 | 0.0441 | 0.0020 | 0.0549* | 0.0217* | 0.4857 |
| SFA | 62.712 | 61.092 | 62.538 | 62.107 | 0.5810 | 0.4856 | 0.4809 | 0.3237 |

¹Values are the mean from n = 8 animals and 3 analysis per animal.
*Means differ (P < 0.10)
Plasma

| | | | | | | | | |
|---|---|---|---|---|---|---|---|---|
| C14:0 | 0.2845 | 0.2808 | 0.3307 | 0.3389 | 0.0130 | 0.8995 | 0.0013* | 0.6551 |
| C16:0 | 13.332 | 13.334 | 13.334 | 12.845 | 0.1302 | 0.5356 | 0.0819* | 0.0805* |
| C17:0 | 0.6461 | 0.6160 | 0.6536 | 0.6386 | 0.0124 | 0.3375 | 0.2434 | 0.5536 |
| C18:0 | 19.784 | 20.195 | 19.020 | 20.492 | 0.6748 | 0.2533 | 0.7340 | 0.4445 |
| C20:0 | 0.4291 | 0.4504 | 0.4630 | 0.4531 | 0.0315 | 0.8782 | 0.5696 | 0.6273 |
| SFA | 34.476 | 34.876 | 33.801 | 34.767 | 0.7228 | 0.5127 | 0.5957 | 0.7009 |

¹Values are the mean from n = 8 animals.
*Means differ (P < 0.10)

The trans fatty acid lipid profiles for milk and blood at are shown in Table 3. Results do not show a significant interaction for any of these fatty acids, however, a significant period effect (P<0.10) exists for the majority of the trans fatty acids in both milk and blood which indicates that, over time, trans fatty acids are reduced. Interestingly, the greatest reduction appears to be observed for the treated goats during the second period. As described above, the periods for the milk analyses span across three weeks and are the average of three analysis, one analysis per week. Period 1 data collection initiates on day 21 post-immunization and ends on day 42, Period 2 data collection initiates on day 49 post-immunization and ends on day 63. However, for the plasma analyses, Period 1 is representative of 42 day post-first immunization and Period 2 is day 63 post-first immunization.

TABLE 3

| | Percent of Total Lipid Profiles¹ | | | | | | | |
|---|---|---|---|---|---|---|---|---|
| | Period 1 | | Period 2 | | | | | |
| Fatty | Neg. | | Neg. | | | | | |
| acid | control | Treated | control | Treated | SE | Treatment | Period | Interaction |

Milk

| | | | | | | | | |
|---|---|---|---|---|---|---|---|---|
| Ct16:1 | 0.4449 | 0.4488 | 0.4195 | 0.4129 | 0.0123 | 0.9597 | 0.0260* | 0.6776 |
| Ct18:1 | 4.3592 | 4.7780 | 3.9056 | 3.8143 | 0.1815 | 0.8106 | 0.0016* | 0.1817 |
| Ctt18:2 | 0.7443 | 0.7832 | 0.6405 | 0.6630 | 0.0174 | 0.6673 | <0.0001* | 0.6440 |
| Cc9t11 | 0.0224 | 0.0206 | 0.0256 | 0.0230 | 0.0040 | 0.5885 | 0.5040 | 0.9190 |
| Trans | 5.5709 | 6.0306 | 4.9912 | 4.9132 | 0.1997 | 0.8033 | 0.0008* | 0.1995 |

¹Values are the mean from n = 8 animals and 3 analysis per animal.
*Means differ (P < 0.10)
Plasma

| | | | | | | | | |
|---|---|---|---|---|---|---|---|---|
| Ct16:1 | 0.3230 | 0.2701 | 0.3250 | 0.2824 | 0.0254 | 0.3315 | 0.7819 | 0.8432 |
| Ct18:1 | 3.7173 | 3.1412 | 3.0939 | 2.7504 | 0.1774 | 0.2011 | 0.0126* | 0.5225 |
| Ctt18:2 | 1.1527 | 1.0165 | 0.8310 | 0.7787 | 0.0390 | 0.4961 | <0.0001* | 0.2995 |
| Trans | 5.1930 | 4.4278 | 4.2499 | 3.8115 | 0.2097 | 0.2152 | 0.0023* | 0.4488 |

¹Values are the mean from n = 8 animals.
*Means differ (P < 0.10)

The results in Table 4 show the non-trans monounsaturated fatty acid profiles for both milk and plasma at Period 1 and Period 2 as described previously for Table 3, in milk, there is a tendency for the fatty acid C14: 1n5 to decrease in milk with treatment. For the fatty acids C18:1n9 and C22:1n9, and total of all measured monounsaturated fatty acids in milk, there are significant or tendencies for significant treatment effects with concentrations increasing in immunized animals compared to the negative control animals. Additionally, for the fatty acids C16:1n7, C18:1n7, and C22:1n9, there are significant or near significant period effects, with C16:1n7 and C18:1n7 being lower in Period 2 than in Period 1 but C22:1n9 being higher in Period 2 than in Period 1. For plasma fatty acids, there are significant or nearly significant Period effects for C14:1n5 and C16:1n7, each showing an increase with time.

TABLE 4

| Percent of Total Lipid Profile¹ | |
|---|---|
| Period 1 | Period 2 |

| | | | | | | | | |
|---|---|---|---|---|---|---|---|---|
| Fatty | Neg. | | Neg. | | | | | |
| acid | control | Treated | control | Treated | SE | Treatment | Period | Interaction |

Milk

| | | | | | | | | |
|---|---|---|---|---|---|---|---|---|
| C14:1n5 | 0.1399 | 0.0973 | 0.1425 | 0.1122 | 0.0207 | 0.0908* | 0.6800 | 0.7695 |
| C16:1n7 | 0.3423 | 0.3685 | 0.1880 | 0.1870 | 0.0147 | 0.3482 | <0.0001* | 0.3709 |
| C18:1n9 | 20.463 | 22.321 | 21.369 | 22.838 | 0.4793 | 0.0559* | 0.1600 | 0.6908 |
| C18:1n7 | 0.0801 | 0.0601 | 0.0582 | 0.0263 | 0.0101 | 0.1897 | 0.0151* | 0.5683 |
| C20:1 | 0.0542 | 0.0591 | 0.0523 | 0.0568 | 0.0045 | 0.3072 | 0.6496 | 0.9675 |
| C22:1n9 | 0.0111 | 0.0132 | 0.0125 | 0.0152 | 0.0008 | 0.0391* | 0.0539* | 0.7195 |
| MUFA | 21.090 | 22.919 | 21.822 | 23.235 | 0.4711 | 0.0631* | 0.2849 | 0.6659 |

¹Values are the mean from n = 8 animals and 3 analysis per animal.
*Means differ (P < 0.10)
Plasma

| | | | | | | | | |
|---|---|---|---|---|---|---|---|---|
| C14:1n5 | 0.1495 | 0.1576 | 0.1711 | 0.1684 | 0.0070 | 0.8382 | 0.0356* | 0.4490 |
| C16:1n7 | 0.3329 | 0.3694 | 0.4375 | 0.4474 | 0.0432 | 0.3473 | 0.0530* | 0.7632 |
| C18:1n9 | 11.244 | 12.551 | 11.384 | 12.086 | 0.4135 | 0.2031 | 0.6999 | 0.4758 |
| C18:1n7 | 0.5627 | 0.5575 | 0.5551 | 0.5003 | 0.0225 | 0.3154 | 0.1728 | 0.2896 |
| MUFA | 12.289 | 13.636 | 12.548 | 13.202 | 0.4529 | 0.2143 | 0.8496 | 0.4569 |

¹Values are the mean from n = 8 animals.
*Means differ (P < 0.10)

Table 5 shows results of milk and plasma non-trans polyunsaturated fatty acid profiles for Period 1 and Period 2 as described previously for Table 3. There is a significant treatment effect for milk fatty acid C20:3n6 showing a decrease with treatment. There is also a significant interaction for plasma fatty acid C20:4n6 demonstrating a decrease with treatment over time in the immunized goats but an increase over time in the negative control goats. Period effects are also observed on other milk and plasma non-trans polyunsaturated fatty acids, with lower percentages observed in Period 2 than in Period 1 for all except C20a4n6 in milk and C18:3n3 in plasma.

TABLE 5

| | Percent of Total Lipid Profiles¹ | | | | | | | |
|---|---|---|---|---|---|---|---|---|
| | Period 1 | | Period 2 | | | | | |
| Fatty | Neg. | | Neg. | | | | | |
| acids | control | Treated | control | Treated | SE | Treatment | Period | Interaction |

Milk

| | | | | | | | | |
|---|---|---|---|---|---|---|---|---|
| 18:2n6 | 5.0852 | 4.5127 | 4.6271 | 4.0441 | 0.1287 | 0.1740 | 0.0029* | 0.9683 |
| 18:3n3 | 0.3149 | 0.2657 | 0.2675 | 0.2208 | 0.0098 | 0.1067 | 0.0003* | 0.8990 |
| 20:3n6 | 0.0335 | 0.0278 | 0.0301 | 0.0263 | 0.0008 | 0.0516* | 0.0065* | 0.2282 |
| 20:4n6 | 0.2090 | 0.2017 | 0.2122 | 0.1925 | 0.0050 | 0.2766 | 0.5579 | 0.2376 |
| PUFA | 5.6426 | 5.0078 | 5.1369 | 4.4838 | 0.1398 | 0.1575 | 0.0025* | 0.9487 |

¹Values are the mean from n = 8 animals and 3 analysis per animal.
*Means differ (P < 0.10)
Plasma

| | | | | | | | | |
|---|---|---|---|---|---|---|---|---|
| 18:2n6 | 36.764 | 35.625 | 31.670 | 31.888 | 0.6290 | 0.7531 | <0.0001* | 0.2989 |
| 18:3n3 | 0.6852 | 0.6804 | 0.7021 | 0.6853 | 0.0252 | 0.7756 | 0.6723 | 0.8151 |
| 20:4n6 | 3.4469 | 3.8132 | 4.0888 | 3.7634 | 0.1287 | 0.9352 | 0.0373* | 0.0177* |
| PUFA | 40.896 | 40.118 | 36.461 | 36.337 | 0.6110 | 0.7455 | <0.0001* | 0.6011 |

¹Values are the mean from n = 8 animals.
*Means differ (P < 0.10)

Volatile fatty acids are measured in the rumen fluid at day 42 (Period 1) and day 63 (Period 2) post-first immunization, and the results are show in Table 6, Results show that treated goats have a significant decrease in propionic acid as compared to non-treated goats. Results also indicate that there is a tendency for an increase in ruminal acetic acid with treatment.

TABLE 6

| | VFA in rumen fluid | | | | | | | |
|---|---|---|---|---|---|---|---|---|
| | (mmoles/100 mmoles¹) | | | | | | | |
| Volatile | Period 1 | | Period 2 | | | | | |
| Fatty Acids | Neg. | | Neg. | | | | | |
| (VFA) | control | Treated | control | Treated | SE | Treatment | Period | Interaction |
| Acetate | 59.767 | 61.070 | 60.224 | 62.073 | 0.6603 | 0.1284 | 0.2880 | 0.6854 |
| Propionate | 16.346 | 14.752 | 17.242 | 13.752 | 0.8335 | 0.0560* | 0.9513 | 0.2744 |
| Iso-Butyrate | 0.9900 | 0.9887 | 1.0825 | 1.2437 | 0.0649 | 0.2276 | 0.0180* | 0.2308 |
| Butyrate | 19.241 | 19.614 | 17.714 | 18.998 | 0.6926 | 0.3624 | 0.1440 | 0.5213 |
| Iso-Valerate | 1.5275 | 1.5800 | 1.7325 | 2.0688 | 0.1383 | 0.2837 | 0.0250* | 0.3222 |
| Valerate | 1.6663 | 1.6075 | 1.6363 | 1.5037 | 0.0929 | 0.3453 | 0.4835 | 0.6975 |

¹Values are the mean from n = 8 animals.
*Means differ (P < 0.10)

In this example, immunizing goats with purified lipase from P. acnes has little observable effect on lipolytic activity, as measured by the rate of free fatty acid accumulation in Period 2. Because ruminal cannulation is a highly invasive technique, oral stomach tubing is used for collection of rumen fluid. Unfortunately, oral stomach tubing for collection of ruminal fluid is not ideal and is prone to saliva contamination. See, Nordlund and Garrett, Bovine Practitioner 28:109-112 (1994). In this example, more than four hours are required to obtain ruminal fluid from all of the goats at each sampling period. Consequently, the ruminal fluid did not arrive back to the laboratory for measurement of lipolytic activity until nearly 4 to 6 hours after dispersal. This delay may possibly explain the high deviation between replications in this assay.

However, evidence for a reduction in lipolytic activity is observed by the significant increase in milk monounsaturated fatty acids in immunized goats compared to negative control goats. With reductions in lipolytic rates, monounsaturated fatty acids remain attached to the glycerol backbone and free from biohydrogenation. Additionally, results also showed that, after immunization, significantly less propionic acid is produced in the rumen. Propionibacterium acnes produces propionic acid as the end product of anaerobic fermentation (Dishisha, et al., Bioresour. Technol. 118:553-562 (2012)). The reduction of propionic acid in the rumen of immunized animals suggests that the immunized animals produce an antibody which may be effectively interfering with the function of this lipolytic bacterium.

These results demonstrate that biohydrogenation is indirectly inhibited by the reduction of lipolytic activity. The milk produced from immunized goats is significantly fortified in non-trans monounsaturated fatty acids, namely, oleic acid (C18:1n9). The immunogenic compositions described herein are effective in reducing milk omega-6 fatty acids, and inhibiting lipolytic activity. Thus, this invention can improve the quality of lipids derived from ruminal animals for human consumption.

### EXAMPLE 6

### Reduction of Fatty Acid Profile of Bovine Milk After Feeding Cows IgY

This experiment demonstrates that, after feeding cows with IgY obtained from eggs inoculated with recombinant P. acnes lipase and with B. fibrisolvens H17C (see Examples 1 and 3 above), the milk fatty acid profile shows more unsaturated fatty acids than occurs in milk from cows not ingesting the IgY. This example involves eight cows total (four in each group) and two periods (replicated) crossover. Two treatments are used; a control (no IgY) and IgY from egg against recombinant P. acnes lipase and B. fibrisolvens H17C. Each test will be ten days. Each group is provided either ground corn (negative control) or ground corn and IgY additive (treatment) to the normal fed-rations.

Samples of each total mixed ration (“TMR”), forages, and concentrates are collected on the last two days of each period and subsequently pooled by period. Feed samples are dried at 55° C. in a forced air oven to determine dry matter (“DM”). After determination of DM, samples are ground to pass a 1 mm screen (Wiley mill, Arthur H. Thomas Co., Philadelphia, Pa.) and are stored at room temperature. Samples of forages and concentrates are analyzed Cumberland Valley Analytical Services, Hagerstown, Md. Analyses include DM (method 4)30,15 (Official Methods of Analysis, 17th ed., AOAC Int., Arlington, Va. (2000))), nitrogen (method #990.03 (Official Methods of Analysis, 18th ed., AOAC Int., Gaithersburg, Md. (2006)); Leco FP-528 Nitrogen Combustion Analyzer, Leco Corp., St. Joseph, Mich.), acid detergent fiber (method #973.18 (Official Methods of Analysis, 17th ed., AOAC Int., Arlington, Va. (2000))), neutral detergent fiber (Van Soest, et al., J. Dairy Sci. 74:3583-3597 (1991)), lignin (Goering and Van Soest., U.S.D.A. Agricultural Research Service, Handbook #379, U.S. Government Printing Office, Washington, D.C. (1970)), starch (Hall, M. B., AOAC Int. 92:42-49 (2009)), either extract using diethyl ether (method #2003.05 (Official Methods of Analysis, 18th ed., AOAC Int., Gaithersburg, Md. (2006)), ash (method #942.05 (Official Methods of Analysis, 17th ed., AOAC Int., Arlington, Va. (2000))) and minerals by inductively coupled plasma (method #985.01 (Official Methods of Analysis, 17th ed., AOAC Int., Arlington, Va. (2000))) except for sulfur which is determined by combustion (Leco S-144DR Sulfur Combustion Analyzer, Leco Corp., St. Joseph, Mich.). Chemical composition of TMR is estimated from analyses of forages and concentrates and their proportion in the diet. Concentration and profile of fatty acids are determined by GC-FID after direct methylation (Sukhija and Palmquist, J. Agric. Food Chem. 36:1202-1206 (1988)) on composite TMR samples using C13:0 or C17:1 (NuChek Prep Inc., Elysian, Minn.) as internal standards as described by Rico, et al., J. Dairy Sci. 97:3739-3751 (2014).

Milk production is measured daily; but measurements from the last 3 days of each period are used to evaluate milk production. Additional milk samples are collected during the morning and afternoon milking on days 8, 9 and 10 and preserved using a pellet of 2-bromo-2-nitropropane-1,3 diol. Milk samples are analyzed for fat, true protein (AOAC, 2000) lactose and solids not fat (“SNF”) using a B2000 Infrared Analyzer (Bentley instruments, Chaska, Minn.) by Heart of America DHIA (Manhattan, Kans.), and milk urea nitrogen (“MUN”) concentration are determined using a modified Berthelot reaction (ChemSpec 150 Analyzer, Bentley instruments, Chaska, Minn.) by the same laboratory using manufacturer's recommended protocol. Yields of milk components are estimated according to milk weight and time of collection. An additional milk sample is taken at the times discussed supra for determination of fatty acids profile. Individual samples are frozen immediately after milking; at the completion of the experiment one composite per cow in each period is obtained by mixing proportional aliquots according to milk weight at the time of collection. Milk fatty acids are analyzed as described by Rico and Harvatine (2013) with modifications. Briefly, milk fat are extracted with hexane:isopropanol, fatty acids (“FA”) methyl esters are prepared by base-catalyzed transmethylation, and FA methyl esters are quantified by GC-FID (20:1 split ratio; initial oven temperature 80° C., increased 2° C./minute to 190° C. and held for 15 minutes, and increased 5° C./min to 215° C. and held for 3 minutes). FA peaks are identified in the gas chromatographic analysis using pure methyl ester standards (GLC 780, 68D, NuChek Prep Inc., Elysian, Minn.; Bacterial Acid Methyl Ester Mix, 47080-U, Sigma-Aldrich Inc., St. Louis, Mo.; GLC-110, Matreya LLC, Pleasant Gap, Pa.). An equal weight reference standard (GLC 461; NuChek Prep Inc., Elysian, Minn.) is used to determine correction factors for individual FA.

Blood is collected at 12 hours on day 10 of each period after feeding via venipuncture of the coccygeal artery into 10-mL evacuated tubes containing K2EDTA (Becton Dickinson and Co., Rutherford, N.J.). Samples are immediately placed in an ice bath and centrifuged within 45 minutes at 3,300×g at ambient temperature for 20 minutes. An aliquot of 4 mL of plasma is deproteinized (4 volumes of plasma are vortexed with 1 volume of 15% sulfosalicylic acid) and then placed in the refrigerator for 10 minutes at 4° C. before centrifuging at 3,300×g at ambient temperature for 20 minutes. The supernatant is collected, and 0.30-mL aliquots are placed into Nunc CryoTube vials (Nalge Nunc International. Roskilde, Denmark) and stored at −20° C. Supernatants obtained from both sampling times will be pooled for each cow during each period. The samples are taken to the laboratory and stored at −80° C. until analyzed.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation. All documents cited herein are incorporated by reference.

We, the inventors, claim as follows:

1. A method of reducing rumen lipolysis in a ruminant comprising administering to said ruminant a lipolysis-inhibiting agent in an amount effective to reduce the amount of lipolysis in the rumen of said ruminant compared to the amount of lipolysis in an untreated ruminant.

2. The method of claim 1; wherein said lipolysis-inhibiting agent comprises one or more different anti-lipolytic antibodies that reduce the amount of lipolysis in the rumen of said ruminant receiving said anti-lipolytic antibodies.

3. The method of claim 2; wherein said anti-lipolytic antibodies binds to an immunogen selected from the group consisting of at least one ruminal bacterium involved in lipolysis, at least one enzyme involved in ruminal lipolysis; at least one co-factor involved in ruminal lipolysis;
and a combination thereof.

4. The method of claim 3; wherein said enzyme involved in lipolysis is lipase.

5. The method of claim 3; wherein said ruminal bacterium is selected from the group consisting of Propionibacterium acnes, Propionibacterium avidum, Anaerovibrio Butyrivibrio fibrisolvens, and a combination thereof.

6. The method of claim 2; wherein said administering to said ruminant is via oral administration.

7. The method of claim 2; wherein said anti-lipolytic antibody is IgY.

8. The method of claim 7; wherein said anti-lipolytic antibody is generated by administering to a hen an amount effective to induce an immune response of an immunogenic composition comprising at least one lipolytic immunogen, a pharmaceutically acceptable carrier, and optionally an adjuvant.

9. The method of claim 8; wherein said lipolytic immunogen is selected from the group consisting of at least one enzyme involved in ruminal lipolysis, at least one co-factor involved in ruminal lipolysis, a fragment of said enzyme, a fragment of said co-factor, at least one ruminal lipase-producing bacterium, and a combination thereof.

10. The method of claim 9; wherein said ruminal lipase-producing bacterium is selected from the group consisting of Propionibacterium acnes, Propionibacterium avidum, Anaerovibrio lipolyticus, Butyrivibrio fibrisolvens, and a combination thereof.

11. The method of claim 9; wherein said enzyme involved in ruminal lipolysis is lipase.

12. The method of claim 1; wherein said lipolysis-inhibiting agent is an immunogenic composition comprising at least one lipolytic immunogen, a pharmaceutical carrier, and optionally an adjuvant; wherein said immunogenic composition induces said ruminant to produce antibodies that bind to said lipolytic immunogen; and wherein said antibodies reduce ruminal lipolysis activity when bound to said lipolytic immunogen.

13. The method of claim 12; wherein said lipolytic immunogen is selected from the group consisting of at least one enzyme involved in ruminal lipolysis, at least one co-factor involved in ruminal lipolysis, a fragment of said enzyme, a fragment of said co-factor, at least one ruminal lipase-producing bacterium, and a combination thereof.

14. The method of claim 13; wherein said ruminal lipase-producing bacterium is selected from the group consisting of Propionibacterium acnes, Propionibacterium avidum, Anaerovibrio Butyrivibrio fibrisolvens, and a combination thereof.

15. The method of claim 13; wherein said enzyme involved in ruminal lipolysis is lipase.

16. The method of claim 12; wherein the route of administration of said immunogenic composition to said ruminant is selected from the group consisting of nasally, orally, sublingually, vaginally, rectally, transmucosally, intramuscularly, intraperitoneally, subdermally, intradermally, subcutaneously, intravenously, and transcutaneously.

17. The method of claim 12; wherein said antibodies are IgA antibodies.

18. A method of enhancing ruminal passage of unsaturated fatty acids and poly-unsaturated fatty acids in a ruminant comprising administering to said ruminant an unsaturated fatty acid protectant in an amount effective to enhance ruminal passage of unsaturated fatty acids and poly-unsaturated fatty acids compared to untreated ruminant.

19. The method of claim 18; wherein said unsaturated fatty acid protectant comprises at least one anti-lipolytic antibody that enhances ruminal passage of said unsaturated fatty acids and said poly-unsaturated fatty acids.

20. The method of claim 18; wherein said an unsaturated fatty acid protectant is an immunogenic composition comprising at least one lipolytic immunogen, a pharmaceutical carrier, and optionally an adjuvant; wherein said immunogenic composition induces said ruminant to produce antibodies that bind to said lipolytic immunogen; and wherein said antibodies enhance ruminal passage of said unsaturated fatty acids and said poly-unsaturated fatty acids when bound to said lipolytic immunogen.

21. A method of reducing the biohydrogenation of unsaturated fatty acids and poly-unsaturated fatty acids in the rumen of a ruminant comprising administering to said ruminant a biohydrogenating-inhibiting agent in an amount effective in reducing biohydrogenation of said unsaturated fatty acids.

22. The method of claim 21; wherein said biohydrogenating-inhibiting agent comprises at least one anti-saturation antibody that reduces the saturation of said fatty acid in said ruminant.

23. The method of claim 21; wherein said biohydrogenating-inhibiting agent is an immunogenic composition comprising at least one hydrogenation immunogen, a pharmaceutical carrier, and optionally an adjuvant; wherein said immunogenic composition induces said ruminant to produce antibodies that bind to said hydrogenation immunogen; and wherein said antibodies reduces ruminal biohydrogenation activity when bound to said hydrogenation immunogen.


SEQUENCE LISTING (6 sequences)

SEQ ID NO: 1 - DNA, 1020 bp, Propionibacterium acnes
atgaagatcaacgcacgattcgccgtcatggccgcgagtgtggccgtcctgatggctgccgcgccgattgcgcaggctgctacttcgccgggggatatccatcccctggtccaggcagcccacagccccgacggtattcccggtaacggcgtcgggccggaattccatacgtcgtcgatggcgcgttcctacagcgagaagcacctgggcgtggcgccgcggggtgtgaacgacttctcctgcaaggtcaagcccggcgaccgaccggtcatcctgattcccggtactggcggcaatgcgttcgccacatggtccttctacggaccccatctggcccatgaggggtactgtgtctacaccttcaccaccaatgtccccgtggggatcctcgacgagggctggggcttcaccggtgacgtgcgcgcctccgcgcaggcgctgggtgcctttgtggatcgggtccggaaagcgacgggctctgagaaggtcgacttcgtcggacattcgcagggtggcggcatcctgccgaacgcctacatcaagatgtatggcggagcgtccaaggtcgacaagctgatcggactggtggccgccaaccacggcaccaccgccgtcggtctcgacaagcttgtcgacggcctgcccgaggctgtgaaggatttccttagcacgtggagctacgaccacaacatggaggcctacggccagcagctcaagggatcagcgttgatgcaacaggtttaccgtgacggcgacaccgtcccgggtatcgcctacaccgtcatctccacccggctcgacatgacggtcacgccctacacgcaagccttcctcaagggcgccaagaacatgaccgtgcaggacgcctgccctctggatgcctatggccacggccgcctaccctatgatcccgtcgcctaccagatggtgctcaacgccctcgatccgaaccacccacgagagatctcctgcacgtggcggccccgggttctgcccgtatccaccacggatgctgcatga

SEQ ID NO: 2 - protein, 339 aa, Propionibacterium acnes
MKINARFAVMAASVAVLMAAAPIAQAATSPGDIHPLVQAAHSPDGIPGNGVGPEFHTSSMARSYSEKHLGVAPRGVNDFSCKVKPGDRPVILIPGTGGNAFATWSFYGPHLAHEGYCVYTFTTNVPVGILDEGWGFTGDVRASAQALGAFVDRVRKATGSEKVDFVGHSQGGGILPNAYIKMYGGASKVDKLIGLVAANHGTTAVGLDKLVDGLPEAVKDFLSTWSYDHNMEAYGQQLKGSALMQQVYRDGDTVPGIAYTVISTRLDMTVTPYTQAFLKGAKNMTVQDACPLDAYGHGRLPYDPVAYQMVLNALDPNHPREISCTWRPRVLPVSTTDAA

SEQ ID NO: 3 - DNA, 942 bp, Propionibacterium acnes
gctacttcgccgggggatatccatcccctggtccaggcagcccacagccccgacggtattcccggtaacggcgtcgggccggaattccatacgtcgtcgatggcgcgttcctacagcgagaagcacctgggcgtggcgccgcggggtgtgaacgacttctcctgcaaggtcaagcccggcgaccgaccggtcatcctgattcccggtactggcggcaatgcgttcgccacatggtccttctacggaccccatctggcccatgaggggtactgtgtctacaccttcaccaccaatgtccccgtggggatcctcgacgagggctggggcttcaccggtgacgtgcgcgcctccgcgcaggcgctgggtgcctttgtggatcgggtccggaaagcgacgggctctgagaaggtcgacttcgtcggacattcgcagggtggcggcatcctgccgaacgcctacatcaagatgtatggcggagcgtccaaggtcgacaagctgatcggactggtggccgccaaccacggcaccaccgccgtcggtctcgacaagcttgtcgacggcctgcccgaggctgtgaaggatttccttagcacgtggagctacgaccacaacatggaggcctacggccagcagctcaagggatcagcgttgatgcaacaggtttaccgtgacggcgacaccgtcccgggtatcgcctacaccgtcatctccacccggctcgacatgacggtcacgccctacacgcaagccttcctcaagggcgccaagaacatgaccgtgcaggacgcctgccctctggatgcctatggccacggccgcctaccctatgatcccgtcgcctaccagatggtgctcaacgccctcgatccgaaccacccacgagagatctcctgcacgtggcggccccgggttctgcccgtatccaccacggatgctgcatga

SEQ ID NO: 4 - protein, 313 aa, Propionibacterium acnes
ATSPGDIHPLVQAAHSPDGIPGNGVGPEFHTSSMARSYSEKHLGVAPRGVNDFSCKVKPGDRPVILIPGTGGNAFATWSFYGPHLAHEGYCVYTFTTNVPVGILDEGWGFTGDVRASAQALGAFVDRVRKATGSEKVDFVGHSQGGGILPNAYIKMYGGASKVDKLIGLVAANHGTTAVGLDKLVDGLPEAVKDFLSTWSYDHNMEAYGQQLKGSALMQQVYRDGDTVPGIAYTVISTRLDMTVTPYTQAFLKGAKNMTVQDACPLDAYGHGRLPYDPVAYQMVLNALDPNHPREISCTWRPRVLPVSTTDAA

SEQ ID NO: 5 - DNA, 39 bp, Artificial Sequence
  forward primer
cgcgaacagattggaggtgctacttcgccgggggatatc

SEQ ID NO: 6 - DNA, 39 bp, Artificial Sequence
  reverse primer
gtggcggccgctctattatgcagcatccgtggtggatac